(19) United States
(12) Patent Application Publication
(10) Pub. No.: US 2021/0391043 A1
(43) Pub. Date: Dec. 16, 2021

(54) METHOD AND SYSTEM FOR FACILITATING INCIDENT REPORTING ASSOCIATED WITH AN EQUINE SHOW

(71) Applicant: SHOW PLUS PROMOTIONS, LLC, Wellington, FL (US)

(72) Inventors: Joey DOMINGUEZ, Wellington, FL (US); Rosemary McAndrew, Wellington, FL (US)

(21) Appl. No.: 17/349,399

(22) Filed: Jun. 16, 2021

Related U.S. Application Data
(60) Provisional application No. 63/039,590, filed on Jun. 16, 2020.

(51) Int. Cl. G16H 10/60; G16H 50/20; G06Q 40/08

(52) U.S. Cl. CPC G16H 10/60; G06Q 40/08; G16H 50/20

(57) ABSTRACT

A method performed by an incident management system for managing or processing incident reporting for, e.g., a benefits program provided by an equine show organizer. The method involves, e.g., receiving, from a registrant user device, an incident report for an incident in which a horse or a rider associated with the registrant suffered an injury or death, generating and sending an information request message which includes a request for diagnosis or treatment information, and receiving and storing the diagnosis or treatment information. The method may further include sending, to the equine show organizer, information associated with the incident report, receiving a benefit payment decision indication from the show organizer, and generating and sending, based on the payment decision indication, a status notification message to a registrant associated with the incident report.

### CROSS-REFERENCE TO RELATED APPLICATION(S)

This application claims the benefit of U.S. Provisional Application No. 63/039,590, entitled “METHOD AND SYSTEM FOR FACILITATING INCIDENT REPORTING FOR AN EQUINE SHOW” and filed Jun. 16, 2020, the entire content of which is incorporated by reference herein in its entirety.

### FIELD OF THE INVENTION

The present invention is directed to an incident management system and method for facilitating the reporting of an incident occurring at an equine competition or, more generally, an equine show.

### BACKGROUND

An equine show (also referred to as a horse show or an equestrian show) may involve an exhibition or competition to demonstrate equestrian skills, such as show jumping, vaulting, eventing, and dressage. The equine show may be organized by a show organizer, and many entries may participate in the equine show. Each entry may include a horse and a rider (the term horse as used herein may include a pony and other types of horses). Each entry may be associated with a registrant, such as a horse owner. The registrant may pay the entrance fee which allows the entry to participate in the equine show.

### SUMMARY

One aspect of the present application relates to an incident management system and/or a method performed by the incident management system. In an embodiment, the incident management system may comprise a communication sub-system, a storage sub-system, and a processing sub-system. The communication sub-system may be configured to receive information from or send information to: (i) a show organizer system or show registration system associated with an organizer of the equine show, (ii) devices of registrants associated with horses that are participating or have participated in the equine show, and (iii) medical service provider systems of medical service providers. The processing sub-system may be in communication with the storage sub-system and the communication sub-system, and may have one or more processing circuits. The one or more processing circuits of the processing sub-system may be configured to: receive via the communication sub-system, from a registrant user device, an incident report for an incident in involving an injury or death for a horse or rider in the equine show. The incident report may include incident description information which describes the incident. The one or more processing circuits may be configured to store the incident report in a database provided on the storage sub-system.

In an embodiment, the processing sub-system may further be configured to generate an information request message which includes a request for diagnosis or treatment information associated with the incident. The diagnosis or treatment information may be requested from a medical service provider that will treat or diagnose the horse or rider for the incident, or that has treated or diagnosed the horse or rider for the incident. The processing sub-system may send the information request message to the registrant's user device via the communication sub-system. The processing sub-system may further receive, via the communication sub-system, after the medical service provider has diagnosed or treated the horse or rider for the incident, the diagnosis or treatment information from a medical service provider system of the medical service provider, and store the diagnosis or treatment information in the database in a manner that associates the diagnosis or treat information with the incident report.

In an embodiment, the processing sub-system may be configured to send, to the show organizer system via the communication sub-system, information from the database that is associated with the incident report. The processing sub-system may receive via the communication system, from a show organizer associated with an organizer of the equine show, a payment decision indication (also referred to as a benefit decision indication or benefit payment decision indication) which indicates approval or denial of payment for the incident under a benefits plan associated with the equine show. The processing sub-system may generate, based on the payment decision indication, a status notification message which indicates whether the incident will be approved or denied payment under the benefits plan.

Features, objects, and advantages of embodiments hereof will become apparent to those skilled in the art by reading the following detailed description where references will be made to the appended figures.

### BRIEF DESCRIPTION OF THE DRAWINGS

The foregoing and other features and advantages of the invention will be apparent from the following description of embodiments hereof as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIGS. 1A-1D provide examples of an incident management system for facilitating reporting of an incident occurring at an equine show, according to an aspect hereof.

FIGS. 2A-2C provide examples of an incident management system, according to an aspect hereof.

FIGS. 3A and 3B depict a flow diagram and workflow of an example method performed by the incident management system, according to an embodiment hereof.

FIG. 4 illustrates a database on a storage sub-system for storing participant information, according to an embodiment hereof.

FIGS. 5A-5C illustrate an example of an incident reporting user interface, according to an embodiment hereof.

FIGS. 6A and 6B illustrate a database of a storage sub-system for storing one or more incident records, according to an embodiment hereof.

FIGS. 7A and 7B illustrate an acknowledgment message and an information request message associated with an incident report, according to an embodiment hereof.

FIG. 7C illustrates a database of a storage sub-system for storing diagnosis and treatment information associated with an incident record, according to an embodiment hereof.

FIG. 8A illustrates example of benefit payment recommendation and other information associated with an incident report that is sent by the incident management system to a show organizer system, while FIG. 8B illustrates an example of a payment decision indication that approves the payment for an incident, according to an embodiment hereof.

FIG. 9A illustrates a workflow involving providing an incident review user interface, while FIGS. 9B-9E illustrate example views of the incident review user interface, according to an embodiment hereof.

FIGS. 10A-10C illustrate an example of a system having a QR code generating system, according to an embodiment hereof.

FIG. 11 depicts an example workflow for managing an incident report, according to an embodiment hereof.

### DETAILED DESCRIPTION

The use of the word “a” or “an” when used in conjunction with the term “comprising” in the patent claims and/or the patent specification may mean “one,” but it is also consistent with the meaning of “one or more,” “at least one,” and “one or more than one.”

The use of the term “or” in the patent claims is used to mean “and/or” unless explicitly indicated to refer only to alternatives or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited, elements or method steps.

One aspect of the present disclosure relates to an incident management system that facilitates incident reporting associated with an incident involving an injury occurring at an equine show, wherein the incident may be reported to seek payments, also referred to as benefit payments, to help with a cost or loss associated with the incident. The payment may be made as part of a benefits plan provided by an organizer of the equine show (also referred to as a show organizer), wherein the payment may be sought by a registrant which registered a horse or rider to participate in the equine show. The cost may include, e.g., out-of-pocket costs not covered by the registrant's own insurance policy associated with treating the injury (if the registrant has such an insurance policy). In an embodiment, the incident management system may be configured to be able to interact with numerous other systems or devices, including registrant user devices, a medical service provider system, a show organizer system, and/or reviewer user devices. For example, the incident management system may be configured to communicate (e.g., over a network) with a registrant, or more specifically with a registrant user device operated by or belonging to the registrant, to receive an incident report from the registrant user device. The incident management system may further be configured to communicate with a medical service provider, or more specifically a medical service provider system operated by or belonging to the medical service provider, to receive diagnosis or treatment information (e.g., treatment report or discharge report) associated with the incident report. The incident management system may further be configured to communicate with a show organizer, or more specifically a show organizer system operated by or belonging to the organizer of the equine show, to send information associated with the incident report to the show organizer's system. The incident management system may thus provide an interface that collects information regarding incidents or incident reports, store the information, and forward the information or a processed version of the information to other systems, thus facilitating an efficient manner of receiving, storing, and otherwise handling incident reports.

In an embodiment, the incident management system may provide a single system and/or user interface for receiving information about incident reports. For instance, the incident management system may provide a single system which is able to communicate with multiple entities, such as a registrant and a medical service provider, to receive the information about incident reports. The incident management system may further use the received incident reports to keep a show organizer updated with respect to incidents occurring at the equine show and incidents for which the show organizer may provide payment as part of a benefits plan (also referred to as a benefit plan). In some situations, the incident management system may belong to or may be operated by an entity which is acting as a benefits plan administrator for the show organizer. In an embodiment, the show organizer may be insured by a captive, also referred to as a captive insurance company. In this embodiment, after the show organizer provides payment for an incident as part of the benefits plan, the show organizer, as an insured, may seek reimbursement from its captive insurance company. If the incident management system belongs to or functions as a benefits plan administrator for the show organizer, the incident management system may in some instances be configured to generate information or other material (e.g., forms, reports, or other documents) which the show organizer may use to seek reimbursement from the organizer's captive insurance company, wherein the reimbursement is for payments made under the benefits plan.

In an embodiment, by interfacing with the multiple entities discussed above, or more specifically with their systems and devices, the incident management system may provide a more automated, centralized, and efficient system for facilitating the reporting of incidents, for storing and presenting information collected from the incident reports so that they can be reviewed, and for sending the information from the incident reports to a show organizer system or another system so as to facilitate decision-making regarding whether to provide benefits (also referred to as payments or benefit payments) under the benefit plan, and/or the amount of a benefit payment for which the registrant may be entitled for an injury suffered by a horse or rider associated with the registrant. As stated above, a show organizer which makes a benefit payment to the registrant may thereafter seek reimbursement from a captive insurance company (also referred to as a captive insurer) which is providing the show organizer with a captive insurance policy for the equine show. In this situation, the incident management system may further facilitate an assessment of whether the show organizer can obtain, from the captive insurance company, reimbursement for that benefit payment. The incident management system may thus provide a technical effect of optimizing how much electronic communication is involved in collecting an incident report and collecting information associated with the incident report, a technical effect of providing an ability of multiple entities (e.g., registrants and medical services providers) to provide information for completing and handling the incident report, and optimizing an ability to provide various entities (e.g., organizer of equine show) with access to information in the incident reports.

In an embodiment, the incident management system may be configured to provide an incident reporting user interface, such as a web portal or webpage, which a registrant may access via a registrant user device, to submit an incident report. In an embodiment, the incident management system may be configured to provide an incident review user interface, such as another webpage, which a reviewer may access via a reviewer user device, to review or perform other tasks associated with handling an incident report. The user interfaces provided by the incident management system may provide a technical effect of providing a centralized system which facilitates efficient collection and handling of incident reports and/or administering of a benefits plan (also referred to as a benefits payment plan) for the reported incidents.

In an embodiment, the incident management system may be configured to generate a task list associated with handling an incident report. For instance, the task list may include scheduled tasks, such as an information request reminder task for sending a reminder to a registrant or medical service provider for diagnosis or treatment information associated with a reported incident. The incident management system may be configured to manage the task list by checking, periodically, whether the task list includes an unfulfilled task which should be performed during a particular time period. Such a functionality of the incident management system may further contribute to the technical effect of providing a centralized system which facilitates efficient collection and handling of incident reports.

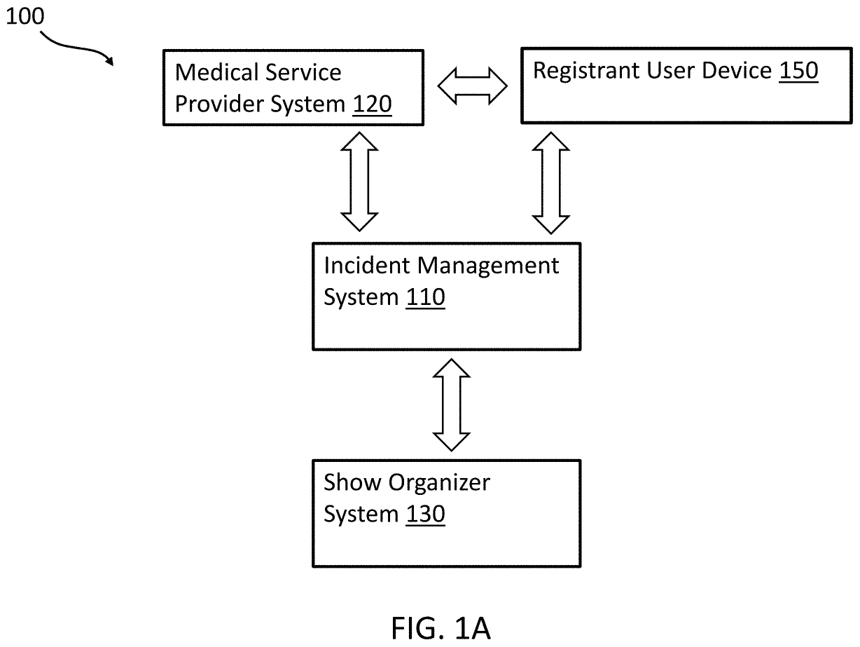

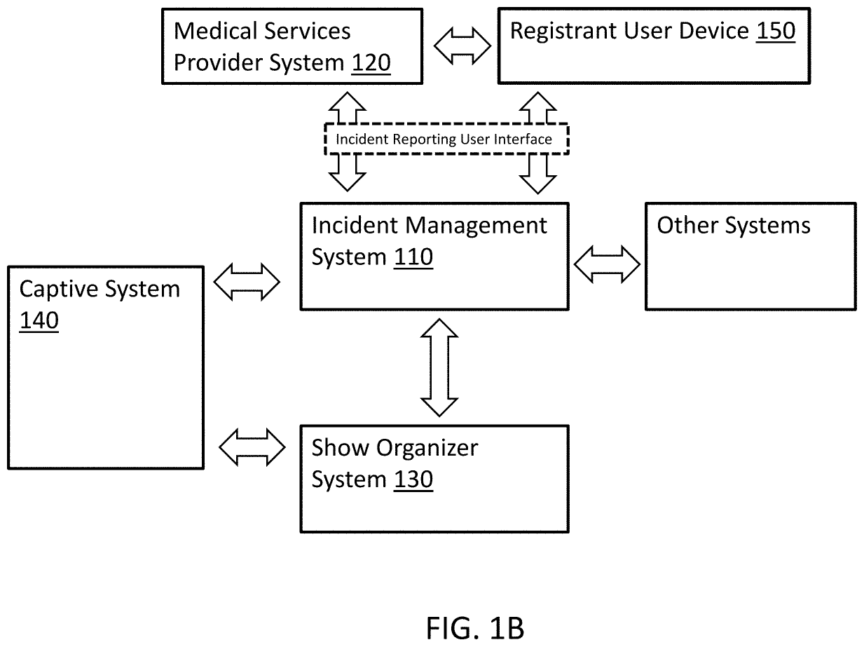

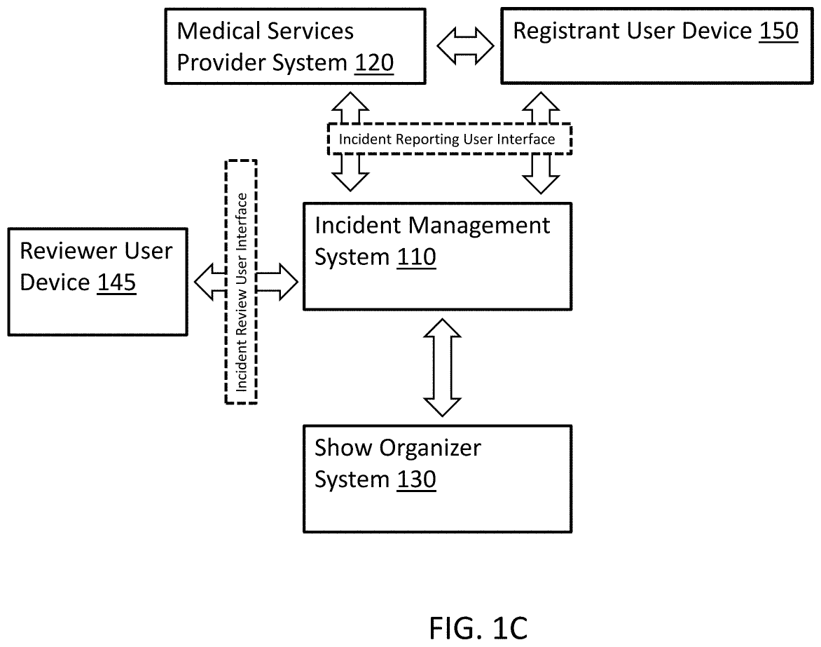

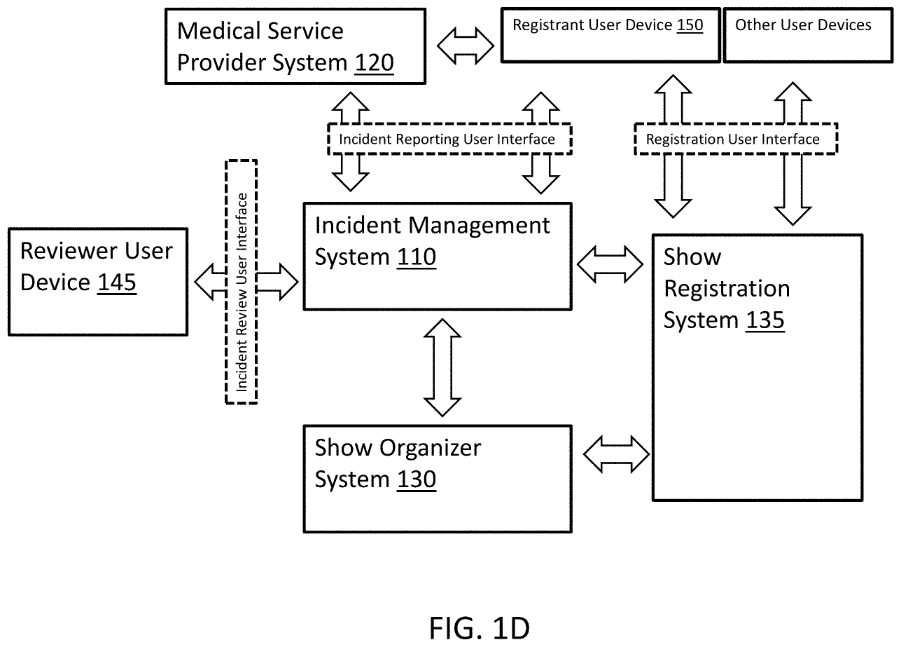

FIG. 1A depicts a system 100 or architecture for facilitating reporting of an incident (e.g., rider injury and/or horse injury) which occurred at an equine show, wherein the incident may be reported to seek payment (also referred to as a benefit payment or benefits payment) for a cost or loss associated with the incident, such as an injury to a horse or rider registered to participate in the equine show. The payment may be made by the organizer of the equine show, and may be sought by a user, or more specifically a registrant (also referred to as a registered user, registered entry, or registered competitor) associated with the injured horse or rider. As stated above, if the organizer is covered by a captive insurance policy provided by a captive insurance company, the organizer may seek, from a captive insurance company associated with the organizer, reimbursement of that benefit payment. Returning to FIG. 1A, the example system 100 includes an incident management system 110, a show organizer system 130, one or more medical service provider systems 120, and one or more user devices, or more specifically one or more registrant user devices 150 belonging to or operated by one or more registered competitors in the equine show. A registrant user device as discussed herein may also be referred to as a registered competitor user device.

In this example, the incident management system 110 may be a computing system for facilitating intake of incident reports and/or the processing or handling of received incident reports. As discussed below, the incident management system 110 may include one or more computing devices, such as one or more servers (e.g., a webpage server and an e-mail server), which is configured to receive incident reports from a user, such as a registered competitor. More particularly, the incident management system 110 may be configured to receive incident reports from the users' devices, such as the registrant user devices 150. Each of the registrant user devices 150 may be an end user device of a registrant (also referred to as registered competitor), who may use the end user device to submit an incident report to the incident management system 110. For example, the end user device may be a computing device such as a laptop, desktop computer, tablet computer, or a mobile phone operated by, belonging to, or otherwise associated with the registrant, who may use the computing device to send the incident report to the incident management system 110. In an embodiment, the registrant may be a person or organization which had registered to have a horse participate in the equine show. The registrant in this example may be, e.g., an owner of the horse participating in the equine show. If the horse and/or a rider suffers an injury or other incident while participating in the equine show, the registrant may submit an incident report to the incident management system 110 via the registrant user device 150 of the registrant. As discussed below, the incident report may identify the horse and/or rider who suffered the injury or other incident, and may include a description of the incident.

In an embodiment, each of the medical service provider systems 120 may be a computing system operated by, belonging to, or otherwise associated with a medical service provider, such as a doctor, veterinarian, clinic, or hospital. The medical service provider may be used by the registrant to provide treatment for a particular incident, or more specifically to diagnose or treat a horse or rider which sustained an injury involved in the incident. Further, after the medical service provider has provided treatment for the incident, it may send a treatment report or other diagnosis or treatment information relating to the incident to the incident management system 110 via the medical service provider system 120. The medical service provider system 120 may include, e.g., one or more end user devices (e.g., laptops, desktop computers, facsimile (fax) machines) and/or one or more servers (e.g., e-mail server).

In an embodiment, the show organizer system 130 may be a computing system operated by, belonging to, or otherwise associated with an organizer of the equine show. The organizer may be, e.g., a person or organization which has organized the equine show. In some instances, the organizer may own, or more generally be associated with, a captive that is intended to reimburse the organizer for payments which the organizer has made for qualifying incidents, such as certain injuries or deaths, which occur during the equine show. In some cases, the show organizer system 130 may include one or more computing devices (e.g., servers) that are able to receive and store registration information from registrants, such as owners of horses that are being registered to participate in the equine show. In some cases, as discussed below with respect to FIG. 1D, the show registration may be collected and stored by a show registration system 135 that is separate from the show organizer system 130. The show organizer system 130 and/or the show registration system 135 may provide a database for storing the registration information, and may include a communication sub-system for sending the registration information to the incident management system 110. In some implementations, the show organizer system 130 and/or the show registration system 135 may be configured to compile the registration information so as to generate show participant information, such as information which includes a list of names of horses and/or riders registered to participate in the equine show, and may be configured to send the show participant information to the incident management system 110. In some implementations, if the show organizer system 130 and/or the show registration system 135 includes a database for storing the registration information, the incident management system 110 may be configured to query the database to determine whether a particular horse in an incident report has been registered to participate in the equine show.

FIG. 1B illustrates an embodiment in which the architecture for facilitating incident reporting may include a captive system 140. In this example, the captive system 140 may be a computing system operated by, belonging to, or otherwise associated with a captive insurance company, also referred to as a captive. As stated above, the captive may be an insurance company that is owned by, controlled by, or otherwise affiliated with an insured entity, such as the organizer of the equine show. The captive may provide a policy which covers the show organizer, or more specifically may reimburse the show organizer after the show organizer has made a benefits payment to a registrant in connection with an injury or other incident. In some implementations, the incident management system 110 may be configured to communicate with the captive system 140, as depicted in FIG. 1B. In other implementations, the incident management system 110 does not communicate with the captive system 140.

In an embodiment, as depicted in FIG. 1B, the incident management system 110 may be configured to provide an incident reporting user interface, such as a web portal on a webpage, for registrants or other users. More particularly, a registrant or other user may access the incident reporting user interface via the registrant user device 150 associated with the registrant (or, more generally, via a user device), and may submit an incident report via the incident reporting user interface. For instance, the incident management system 110 may include a webpage-hosting server configured to generate content, such as HTML, CSS, and form information, used to generate the incident reporting user interface, and configured to send the content to the registrant user device 150. The registrant user device 150 may use the content to display the incident reporting user interface, such as a webpage having one or more forms for entering incident description information. In this example, the registrant may provide incident description information as user input via the registrant user device 150 to the one or more forms of the incident reporting user interface. The registrant user device 150 may send this user input, or more specifically the incident description information, to the incident management system 110.

FIG. 1C depicts an example in which the incident management system 110 may be configured to provide an incident review user interface for reviewing incident reports, wherein the reviewing of the incident reports may be part of a process of handling the incident reports. For example, the incident review user interface may be a web portal or other webpage which may present information about incident reports received by the incident management system 1100. In such an example, the incident management system 110 may include one or more webpage-hosting servers for hosting one or more webpages. The one or more servers may, e.g., both host an incident reporting webpage to be used by registrants to submit incident reports, and host an incident review webpage to be used by reviewers or other personnel for reviewing the incident reports. The incident reporting webpage may be a front-end to the incident management system 110 for interfacing with registrants, while the incident review webpage may be part of a back-end of the incident management system 110 for handling incident reports. The reviewers of the incident reports may access the incident review user interface via reviewer user devices 145. Each of the reviewer user devices 145 may be a computing device, such as a laptop, tablet computer, or mobile phone that is operated by or otherwise associated with a reviewer of incident reports. The server in this example may send content, such as stored incident description information along with HTML and CSS, to the review user device 145, which may use the content to display the incident description information within the incident review user interface.

As discussed above, registration information for an equine show may be received by the show organizer system 130, and/or by a show registration system 135 illustrated in FIG. 1D. In the example of FIG. 1D, the show registration system 135 may present a registration user interface, such as a webpage which users can access via their user devices to register for an equine show. A registrant seeking payment for an incident in this example may be one of the users, and may have registered for the equine show using the registration user interface. The show registration system 135 in this example may be configured to store registration information in a database provided within the show registration system 135, and/or may be configured to send the registration information to the show organizer system 130, and/or to the incident management system 110.

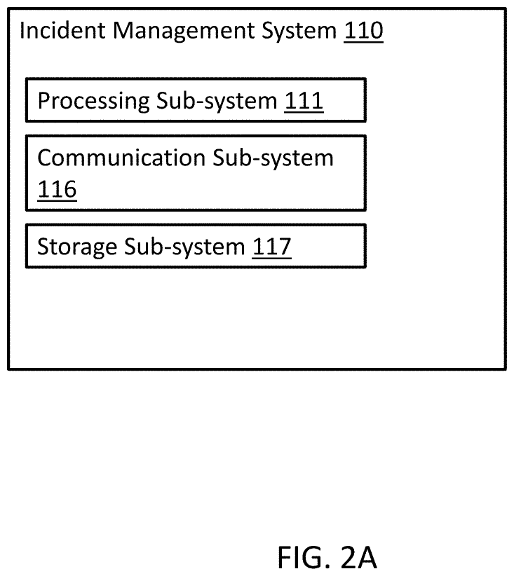

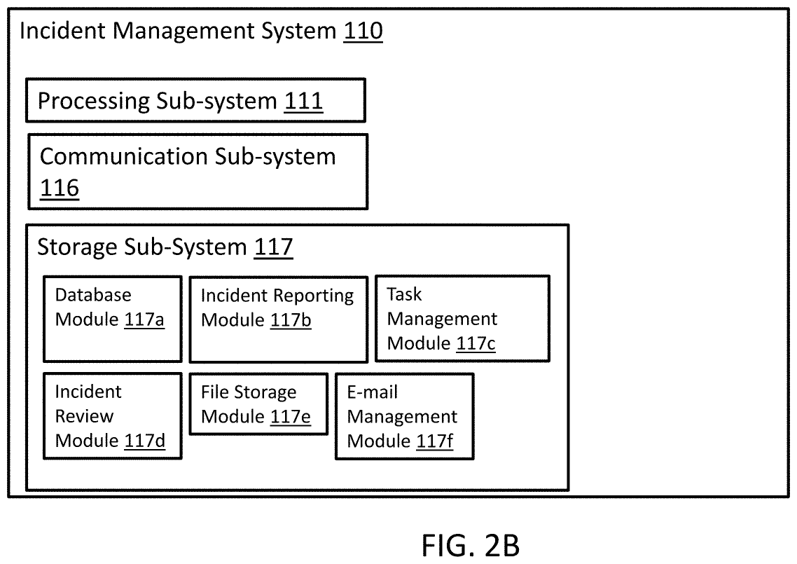

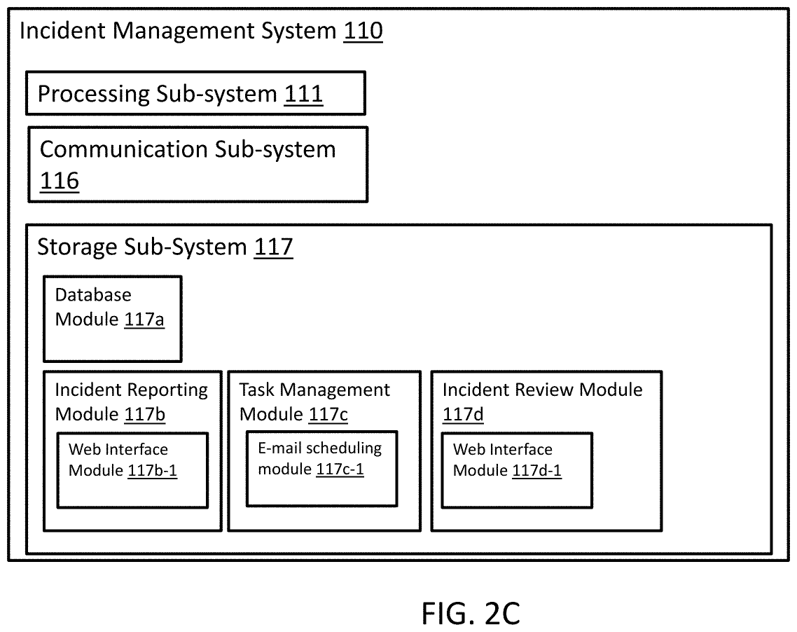

FIG. 2A depicts an example of an incident management system 110. In an embodiment, the incident management system 110 may belong to an entity which interfaces with an organizer of an equine show, with registrants seeking payment for incidents occurring at the equine show, and/or with medical service providers who have provided treatment or will provide treatment for the incidents. More particularly, the incident management system 110 may interface with a show organizer system 130, show registration system 135, registrant user devices, medical service provider systems 120, and/or reviewer user devices 145 (as illustrated in FIGS. 1A-1D) via one or more networks, such as the Internet. By interfacing with these systems and devices, the incident management system 110 may provide a more automated and efficient system for facilitating the reporting of incidents, for storing and presenting information collected from the incident reports so that they can be reviewed, and for sending the information from the incident reports to the show organizer system so as to facilitate decision-making regarding whether to provide payment under a benefits plan for an injury or other incident, and how much payment to provide.

In an embodiment, the incident management system 110 may be a computing system that includes one or more computing devices, such as one or more desktop computers, servers (e.g., e-mail servers, webpage servers), or other computing devices. As depicted in FIG. 2A, the incident management system 110 may include a processing sub-system 111, a communication sub-system 16, and a storage sub-system 117. In an embodiment, the communication sub-system 116 may be a component of the incident management system 110 for receiving and/or sending information, requests, or other communication between the incident management system 110 and the other systems in FIGS. 1A-1D. For instance, the communication sub-system 116 may include one or more network interface components, such as a modem, router, and/or Ethernet communication circuit, which is configured to communicate with other devices and systems via the Internet or other network.

In an embodiment, the storage sub-system 117 may be a component of the incident management system 110 for storing information received or generated by the incident management system 110. For instance, the storage sub-system 117 may include one or more storage devices, such as a hard disk drive (HDD), a solid state drive (SSD), a tape drive, or some other storage device.

In an embodiment, the processing sub-system 111 may be a component of the incident management system 110 for performing various operations on information received via the communication sub-system 116, such as operations for verifying the information and/or transferring the information to storage on the storage sub-system 117. The processing sub-system 111 may include, e.g., one or more processing circuits, such as one or more microprocessors, also referred to as processors (e.g., CPU's). If the incident management system 110 includes multiple computing devices (e.g., multiple servers), the microprocessors may be distributed across the multiple computing devices.

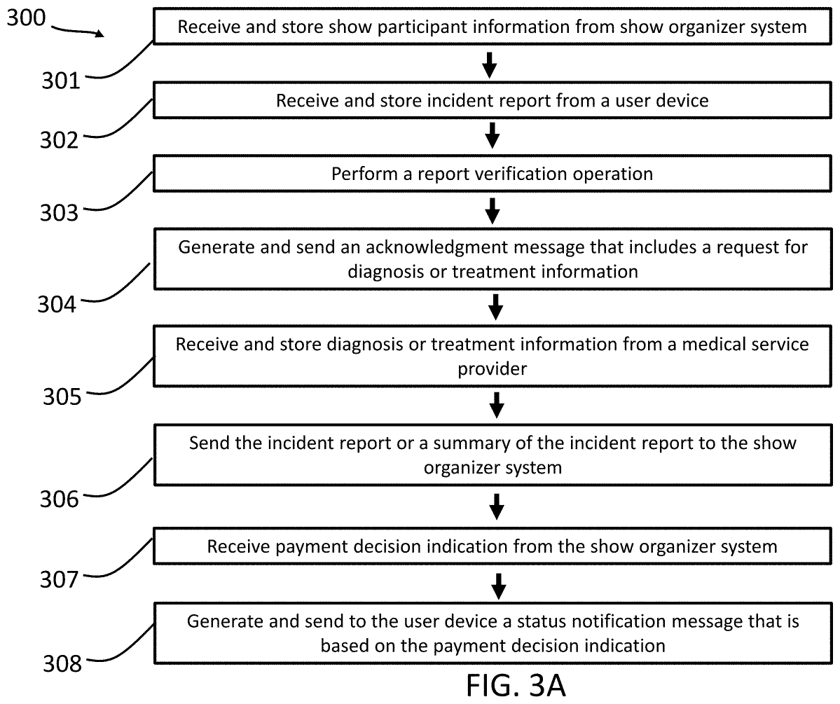

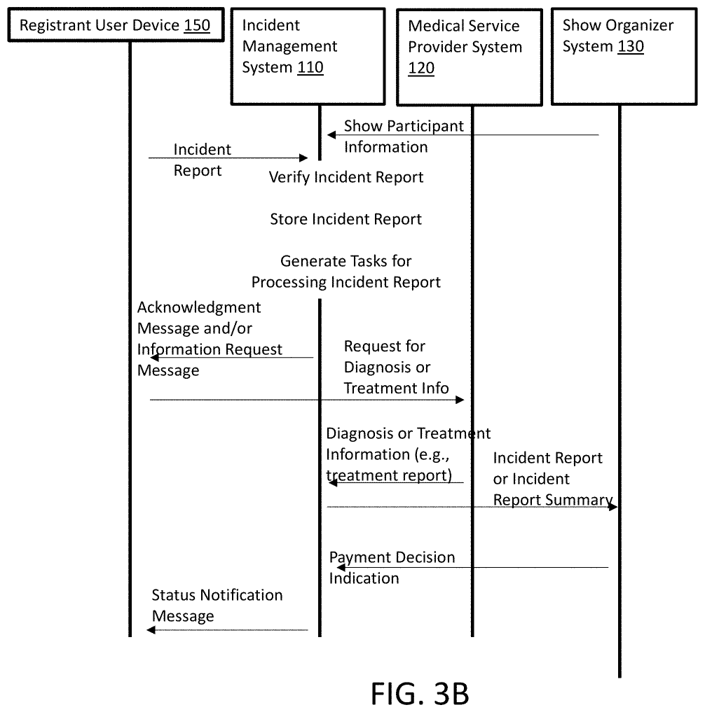

FIG. 2B depicts an example in which the incident management system 110 may be configured to execute various modules, such as a database module 117a, an incident reporting module 117b, a task management module 117c, an incident review module 117d, a file storage module 117e, and an e-mail management module 117f. The modules may be, e.g., software modules that are stored as computer-executable instructions on the storage sub-system 117, and may be executed by the processing sub-system 110. In some instances, these modules may be used to perform various operations, such as the operations described in the method of FIGS. 3A and 3B, which are discussed below in more detail.

In some implementations, the database module 117a may be configured to store incident description information, show participant information, treatment reports or discharge reports (or, more generally, diagnosis or treatment information), and/or other information received by the communication sub-system 116 of the incident management system 110. The database module 117a may, e.g., a customer relationship management (CRM) module that provide a database that organizes the incident description information and show participant information. For example, the database may associate incident description information with treatment reports that pertain to the same incident or the same registrant. In an embodiment, the database module 117a may store a status of an incident report, such as whether the incident report is undergoing review, whether the incident report has been processed, whether follow-up is needed for the incident report, whether payment for the reported incident under a benefits plan is approved, or whether payment for the reported incident is denied.

In some implementations, the incident reporting module 117b may be configured to communicate with registrant user devices 150 so as to receive incident reports from the registrant user devices 150. For instance, the incident reporting module 117b may be configured to generate content for a incident reporting user interface. More particularly, the incident reporting module 117b may in an example include a web interface module 117b-1 that is configured to generate web content (e.g., HTML and form content) for an incident reporting webpage, so that the web content can be communicated to registrant user devices via the communication sub-system 116. The incident management system 110 in this example may receive an incident report from a registrant user device 150 via the incident reporting webpage.

In an embodiment, the task management module 117c of FIG. 2B may be configured to generate a list of tasks which should be performed to handle and dispose of an incident report. For instance, the lists of tasks may include an information request task that requests diagnosis or treatment information relating to an incident, an information request reminder task that reminds a registrant to provide the diagnosis or treatment information directly or to prompt a medical service provider to provide that information (if such information has not yet been received), a recommendation task for generating a recommendation based on the diagnosis or treatment information, wherein the recommendation is for whether to make a payment (also referred to as a benefit payment or benefits payment) for a cost or loss associated with the reported incident, and/or other tasks associated with handling an incident report. In some implementations, the task list may include scheduled tasks. For example, the information request reminder task may be scheduled to occur a predefined amount of time (e.g., 2 weeks) after receiving the incident report, or after an initial information request message has been sent. As discussed below, the information request reminder task may be associated with a condition for performing the task, such as a condition involving the incident management system 110 not having yet received the requested information.

In some implementations, the task management module 117c may assign each of the tasks in the task list to a person or system, by associating each of the tasks with a person or system. For example, the task management module 117c may assign a first task in the list to a reviewer, and assign a second task in the list to the incident management system 110 itself, wherein the reviewer in this example may be a person tasked with reviewing incident reports received by the incident management system 110. In some instances, the information request task and the information request reminder task are assigned to the incident management system 110. In such an implementation, the management module 117c may include an e-mail scheduling module 117c-1, as depicted in FIG. 2C, that is configured to generate e-mails and send e-mails to fulfill such tasks. More particularly, the e-mail scheduling module 117c-1 may be configured to generate and send a first e-mail which requests diagnosis or treatment information, and configured to check, at a scheduled time, whether the requested information has been received. If the requested information has not yet been received at that time, the e-mail scheduling module 117c-1 may be configured to generate and send a second e-mail which provides a reminder for the diagnosis or treatment information. In some implementations, the task management module 117c may be configured to delete, from the task list, tasks that have been completed or no longer need to be completed.

In an embodiment, the e-mail management module 117f may be configured to manage e-mails that are sent or received by the incident management system 110, including e-mails for requesting diagnosis or treatment information. The diagnosis or treatment information may be requested from a medical service provider that will treat or has treated a rider or horse which suffered an injury or other incident. For instance, the e-mail management module 117f may be configured to track e-mails relating to incident reports have been sent out via the incident management system 110 (e.g., sent to registrant user devices), and e-mails relating to incident reports received by the incident management system 110 (e.g., received from the medical service provider, or more specifically from the medical service provider system 120). The module 117f may be configured to link e-mails in a forward and backward manner, so that if one e-mail is a reply to a previous e-mail, the two e-mails may be linked to each other.

In an embodiment, the incident review module 117d of FIGS. 2B and 2C may be configured to generate an incident review user interface that presents various information included in or associated with incident reports received by the incident management system 110. More particularly, the incident review module 117d may generate content for the incident review user interface, and send the content to a reviewer user device 145, which may be configured to display the content. For instance, FIG. 2C depicts an example in which the incident review module 117d includes a web interface module 117d-1 that is configured to generate web content (e.g., HTML content) for an incident review webpage, and to send the web content to the reviewer user device 145.

In an embodiment, the file storage module 117e may be configured to manage files that are associated with received incident reports. The files may include, e.g., treatment reports, discharge reports, or other diagnosis or treatment information associated with an incident described in an incident report, include receipts associated with diagnosing or treating the incident, or include other paperwork associated with the incident. The file storage module 117e may be configured to communicate with a reviewer user device 145 to identify files which are available for access by a reviewer of the device 145, including files which the reviewer user device 145 can download or upload. In some implementations, the file storage module 117e may be configured to store files on a third party platform, such as DropBox®, and to retrieve files from the third party platform.

FIG. 3A illustrates an example method 300 which may be performed by, e.g., the incident management system 110 as part of facilitating the reporting of incidents which occur at an equine show. As stated above, the incidents may be reported by a registrant or other user as part of seeking payment under a benefits plan provide by an organizer of the equine show. In some instances, the plan may be referred to as a benevolent benefits plan, and may be backed by a captive insurance policy associated with the organizer of the equine show. The registrant or other user or entity submitting the incident report may be associated with a horse or horses that are participating or have participated in the equine show. In some instances, the method 300 may include steps that are performed by the processing sub-system 111 of the incident management system 110, such as when the processing sub-system 111 executes instructions stored on the storage sub-system 117 (also referred to as a non-transitory computer-readable medium).

In an embodiment, as depicted in FIG. 3A, the method 300 may include a step or operation 301, in which the incident management system 110 receives show participant information which identifies horses or riders registered to participate in an equine show. The show participant information may be received, e.g., from the show organizer system 130 or the show registration system 135 of FIG. 1D via the communication sub-system 116. In some instances, the show participant information may include or may have been generated based on registration information submitted to the show organizer system 130 or to the show registration system 135 from various user devices. For example, the registration information may have been submitted to the show organizer system 130 or the show registration system 135 via a registration user interface, and then stored in a database of the system 130/135. In this example, the incident management system 110 may receive the show participant information from the database of the system 130/135.

In an embodiment, step 301 may include storing the show participant information in a database of the incident management system 110. For instance, the database may be provided on the storage sub-system 117 of the incident management system 110, and more particularly may be provided by the database module 117a (e.g., CRM module) of FIGS. 2B and 2C.

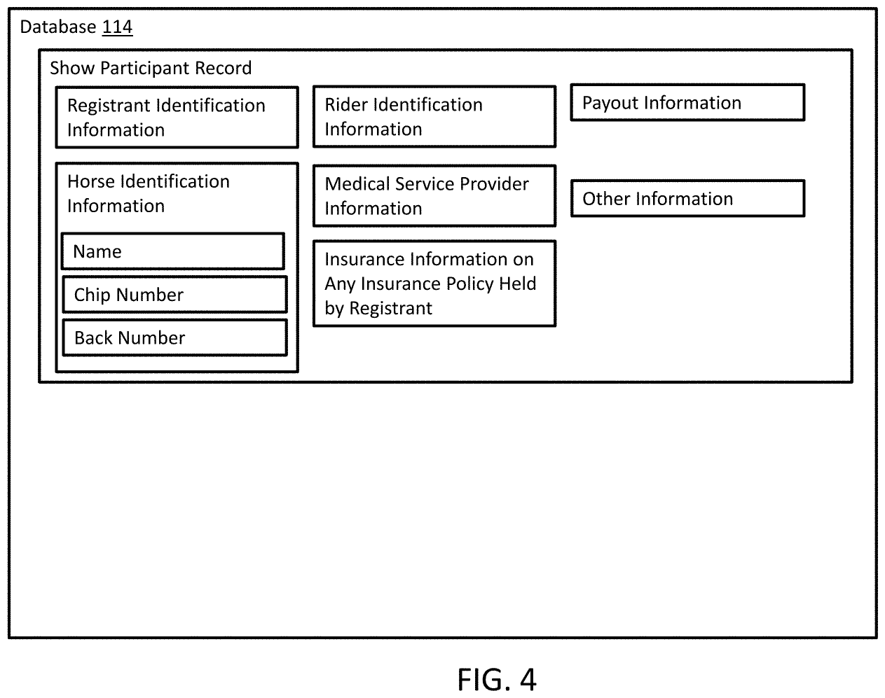

FIG. 4 illustrates a database 114 in which the show participant information may be stored as part of step 301, wherein the database 114 may be provided by, e.g., the database module 117a. In this example, the show participant information may include show participant records, wherein each participant record may be associated with a particular horse registered in an equine show. As illustrated in FIG. 4, the show participant record may include horse identification information, rider identification information, and registrant identification information. The horse identification information may identify a horse which has been registered to participate in an equine show. For instance, the horse identification information may include a horse name, a chip number, and/or a back number. The rider identification information may identify a rider of the horse. For example, the rider identification information may include a name of the rider. In an embodiment, the registrant identification information may identify a particular registrant to an equine show, wherein the registrant may be, e.g., an owner of the horse which has been registered to participate in the equine show.

In some implementations, the show participant record in the database may store other information, such as medical service provider information, insurance information, and/or payout information. The medical service provider information may identify a medical service provider (e.g., hospital, clinic, physician, and/or veterinarian) already used or otherwise selected by the registrant to provide medical service for the horse and/or rider during the equine show. The insurance information may indicate whether the registrant has an insurance policy. In some cases, the registrant may have an insurance policy (e.g., a commercial insurance policy). In such cases, the benefits plan provided by the organizer of the equine show may be in excess of an amount what is covered by the commercial insurance policy (if any) of the registrant.

In an embodiment, the payout information may indicate whether a previous payout has been made by the show organizer to the registrant under, e.g., a benevolent benefits plan. For example, if the registrant had previously submitted an incident report at the same equine show or at an earlier equine show where the benefits were also provided, and had received a corresponding payout of benefits, the payout information may indicate the previous payout.

Returning to FIGS. 3A and 3B, the method 300 may include a step 302, in which the incident management system 110 receives an incident report from a registrant, or more specifically from a registrant user device 150 (e.g., laptop or mobile phone) of the registrant. More generally, the incident management system 110 may receive an incident report from a user device of a user. If the user has registered a horse or rider to participate in the equine show, the user is then a registrant. The incident report from the user may be used to seek payment for an incident in which a horse or a rider associated with the user has suffered an injury or death, and may include incident description information which describes the incident.

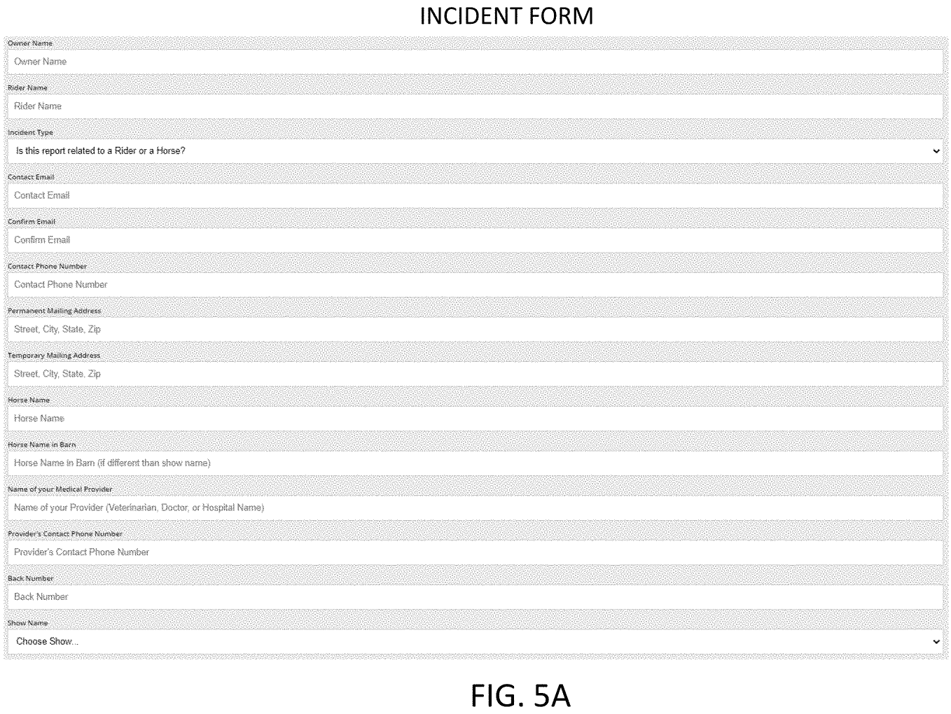

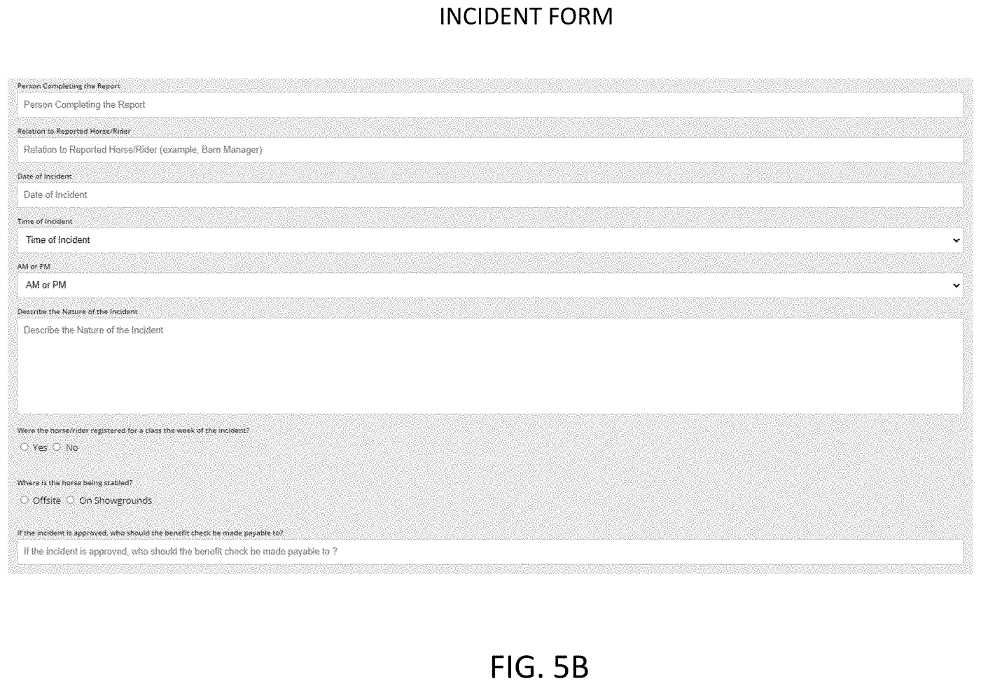

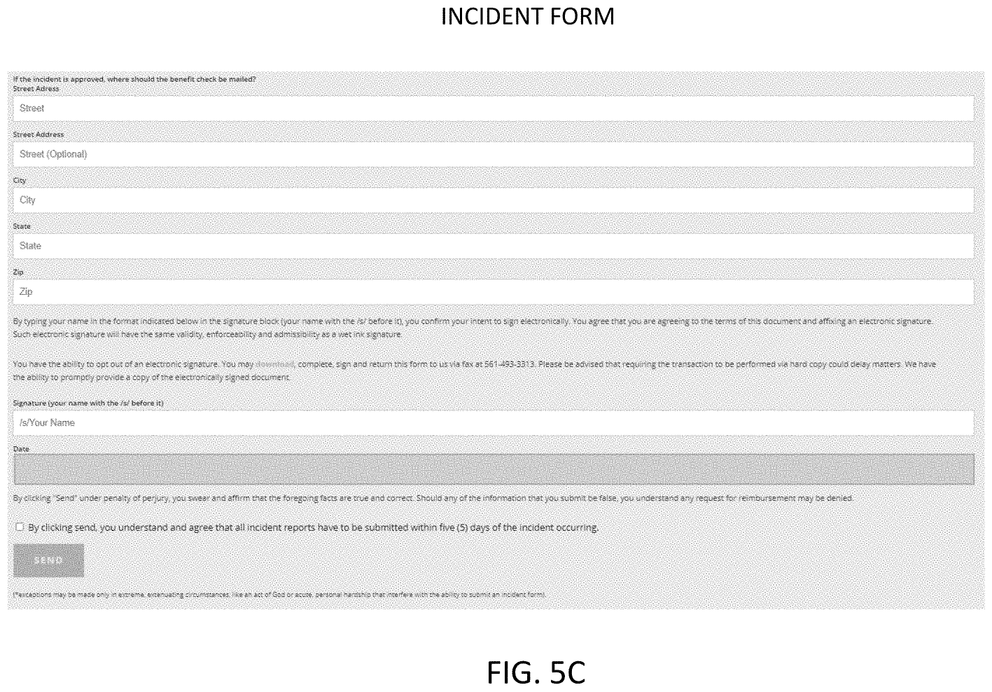

In an embodiment, the incident report may be received via an incident reporting user interface, such as an incident reporting webpage. For example, FIGS. 5A-5C illustrate an example user interface that includes content which may be communicated by the incident management system 110 to a registrant user device 150, and then displayed as a graphical user interface (GUI) on the registrant user device 150. The content may include, e.g., a form having multiple fields associated with different categories of information relating to an incident. As illustrated in FIGS. 5A-5C, the multiple fields may be associated with owner identification information for a horse registered in the equine show (e.g., the owner may be the registrant submitting the incident report), horse identification information, rider identification information, incident type, contact information for the registrant, medical service provider associated with the registrant, show information that identify the equine show at which the incident occurred, date and time at which the incident occurred, a summary of the incident, and other incident description information. The registrant or someone on behalf of the registrant may submit an incident report via the user interface of FIGS. 5A-5C, and the incident management system 110 may receive the incident report from the registrant via the user interface.

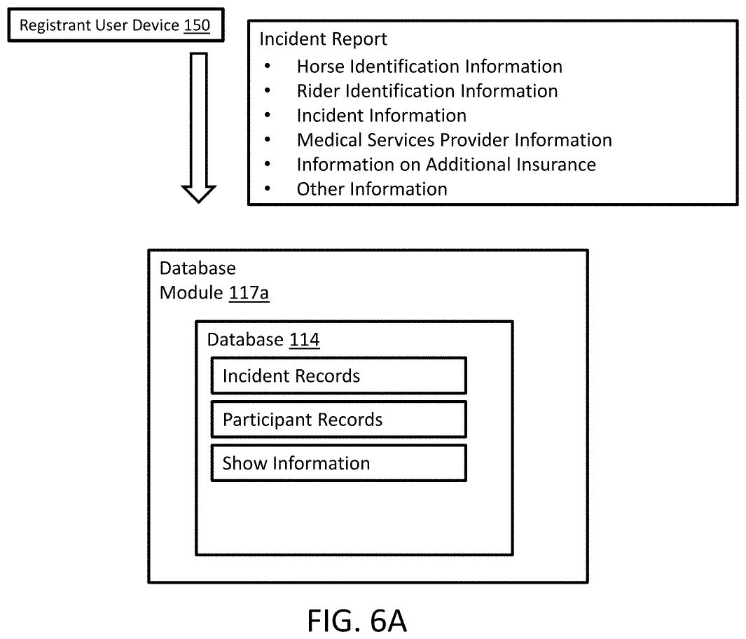

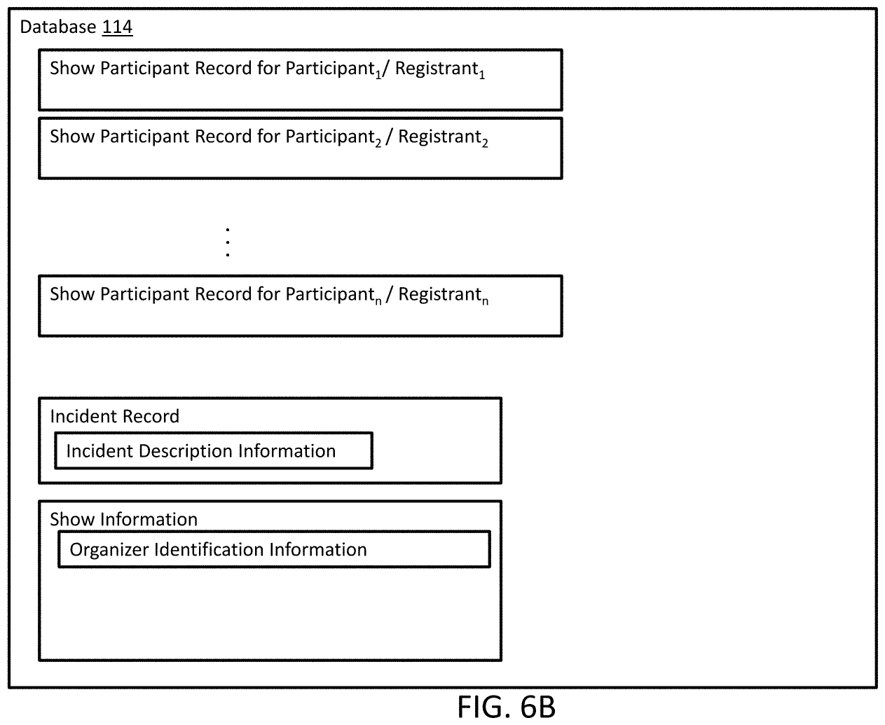

In an embodiment, step 302 may include storing the incident report in the database of the incident management system 110, such as the database provided on the storage sub-system 117. More particularly, FIG. 6A illustrates the database module 117a of the incident management system 110 receiving incident description information in an incident report from a registrant user device 150, and storing the incident description information as an incident record in the database 114. FIG. 6B illustrates an example of the database 114, which may include the show participant record received from the show organizer system 130, and include the incident description information (e.g., owner identification information, horse identification information, rider identification information, type of incident, date and time of incident, incident location, incident summary) received from the registrant user device 150.

In an embodiment, the incident report may omit various fields of incident description information, because that information may be available from the show participant information. For instance, the incident report may omit a field(s) relating to the horse identification information, and/or a field(s) relating to the rider identification information, because such information may have already been provided as part of the show participant information from the show organizer system 130. In some scenarios, the incident report may omit a field(s) relating to a medical service provider associated with a registrant, because the registrant may not have yet selected a medical service provider for treating an incident at the equine show.

FIG. 6B depicts an example in which the database 114 of the incident management system 110 further includes show information, which may describe an equine show associated with incident reports being received by the incident management system 110. For instance, the show information may include organizer identification information to identify an organizer of the equine show.

Returning to FIGS. 3A and 3B, the method 300 may include a step 303 in which the incident management system performs a report verification operation to verify whether a horse which suffered an incident in the incident report was registered with the equine show, or whether a rider who suffered an incident is associated with a horse that was registered with the equine show. The report verification operation may involve comparing the horse or rider described in the incident report with the show participant information to determine whether the horse or the rider in the incident report matches one of the horses or riders identified in the show participant information. If the incident management system 110 identifies a match, it may determine that the horse or rider in the incident report has been registered to participate in the equine show, and may then proceed with other steps for handling the incident report. If the incident management system 110 fails to identify a match, it may determine that the horse or rider in the incident report has not been registered to participate in the equine show. In some instances, the incident management system 110 may determine that a lack of registration for the horse may disqualify an incident in the indent report from being qualified for payment under a benefits plan provided by the organizer of the equine show. In such instances, the incident management system 110 may send, to the registrant user device 150, a status notification message which indicates that the incident report has failed the report verification operation, and/or indicates that the incident report is automatically being denied payment because it has failed the report verification operation.

In an embodiment, the report verification operation may involve determining whether the received incident report has missing information. More particularly, if the incident report is received via the incident reporting user interface (e.g., incident reporting website) having multiple form fields for collecting different categories of information about tan incident, the incident management system 1100 may determine whether the incident report is missing information in at least one of the form fields. If the incident report is missing information in at least one of the form fields, the incident management system 110 may be configured to send a request to the registrant user device to provide the information that is missing.

In an embodiment, the incident management system 110 may be configured to generate a task list in response to receive an incident report, and/or in response to the incident report passing the report verification operation. For instance, the task management module 117c may generate a task list that includes a plurality of tasks associated with handling an incident report. As stated above, the plurality of tasks may include an information request task and an information request reminder task. The task management module 117c may keep track of which tasks have been fulfilled and which tasks are still unfulfilled. In some instances, some or all of the unfulfilled tasks may have a condition for performing the unfulfilled task.

For example, the information request task may involve sending a message (e.g., e-mail) to a registrant's user device to indicate that diagnosis or treatment information is needed from a medical service provider treating an incident in the incident report, while the information request reminder task may involve sending a reminder message if the diagnosis or treatment information has not yet been received. The condition for performing the information request task may involve the incident management system 110 having received an incident report, and/or the incident report having successfully passed a report verification operation, which is discussed above. In some instances, the condition for performing the information request reminder task may involve the incident management system 110 not having yet received the requested information (e.g., the diagnosis or treatment information), and a deadline for the requested information arriving within a predefined amount of time (e.g., in 1 week). In some instances, if the task management module 117c is configured to schedule tasks in the task list, then the condition for the information request reminder task may involve the incident management system 110 not having yet received the requested information, and the arrival of a scheduled time associated with the task. The task management module 117c may be configured to track whether a condition for an unfulfilled task is satisfied. In some instances, the task management module 117c may be configured to track whether an unfulfilled task is no longer necessary, such as when a condition associated with the task can no longer be satisfied. For example, if the incident management system 110 has received a treatment report or other information associated with an information request before a time for sending a reminder has arrived, then it may no longer be necessary to perform the information request reminder task, because the condition for performing the information reminder task can no longer be satisfied.

In an embodiment, the incident management system 110, or more specifically the task management module 117c, may be configured to manage a task list by assigning each task on the task list to either one or more reviewers (e.g., personnel responsible for reviewing incident reports), to the incident management system 110, or both. The incident management system 110 may determine, periodically (e.g., every hour, every day, or every week), whether the task list includes an unfilled task, and whether a condition for performing the unfilled task is satisfied. For instance, as discussed above, the condition for performing the information request reminder task may involve the incident management system 110 not having yet received diagnosis or treatment information associated with an incident report, and a deadline for the diagnosis or treatment information arriving within a predefined amount of time (e.g., the deadline will pass in one week). If the condition is satisfied (e.g., the deadline is within a predefined amount of time, and the diagnosis or treatment information has not yet been received), the incident management system 110 may determine that the information request reminder task should be performed. If the condition has not been satisfied (e.g., the diagnosis or treatment information has already been received, or there is more than a predefined amount of time before arrival of the deadline for the information) the system 110 may determine that the unfulfilled task should not be performed at all, or should not be performed yet.

In an embodiment, the incident management system 110 may determine whether an unfulfilled task is assigned to the incident management system 110 or to one or more reviewers. If the unfulfilled task is assigned to the incident management system 110, the system 110 may perform the unfulfilled task. For example, if the unfulfilled task is an information request reminder task, the system 110 may perform the task by sending an information request reminder message (e.g., reminder e-mail) to a registrant user device 150, to remind the registrant of the device 150 to follow up with a medical service provider so that the medical service provider can provide diagnosis or treatment information to the incident management system 110 by a particular deadline. If the unfulfilled task is assigned to one or more reviewers of incident reports, the incident management system 110 may send a task reminder message to one or more reviewer user devices 145 of the one or more reviewers. For example, the task reminder message may be an e-mail to the one or more reviewer user devices 145, to remind the one or more reviewers to perform a particular task.

In an embodiment, the incident management system 110 may be configured to provide an incident review user interface for a reviewer of incident reports. The reviewer may be, e.g., a person responsible for performing various tasks associated with handling the incident reports. In this embodiment, the reviewer may view the incident review user interface via a reviewer user device 145 (e.g., laptop or mobile phone) of the reviewer. In some instances, the incident review user interface may be a GUI, such as a webpage, for presenting information relating to incident reports received by the incident management system. In some instances, the incident review user interface may present the task list discussed above.

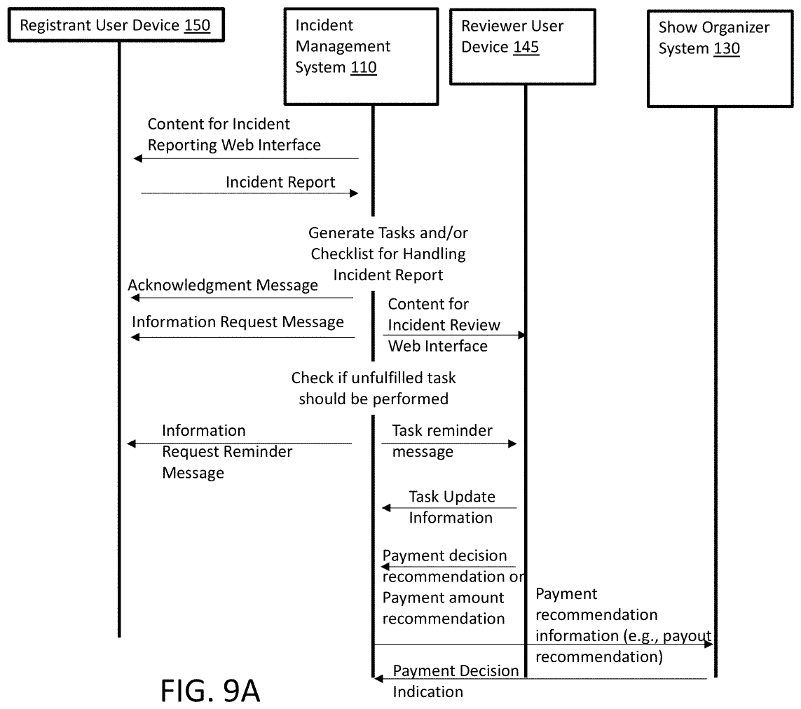

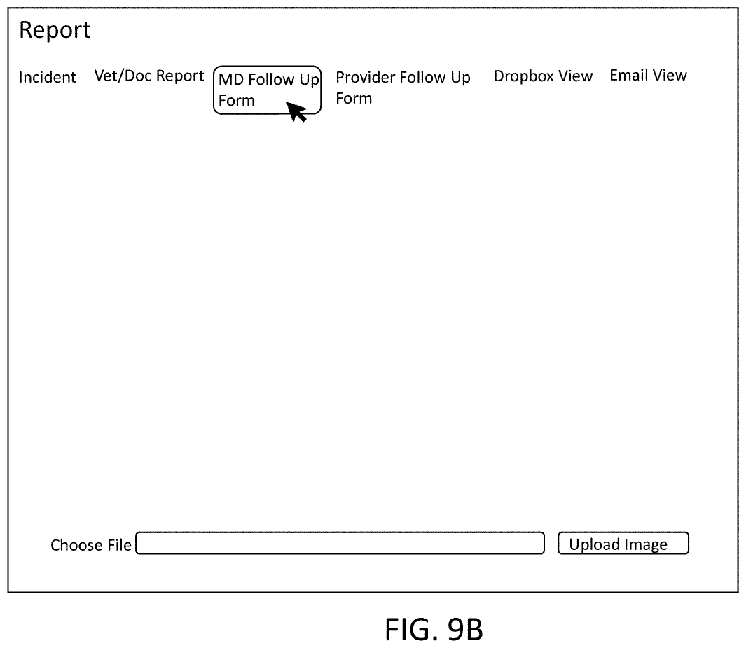

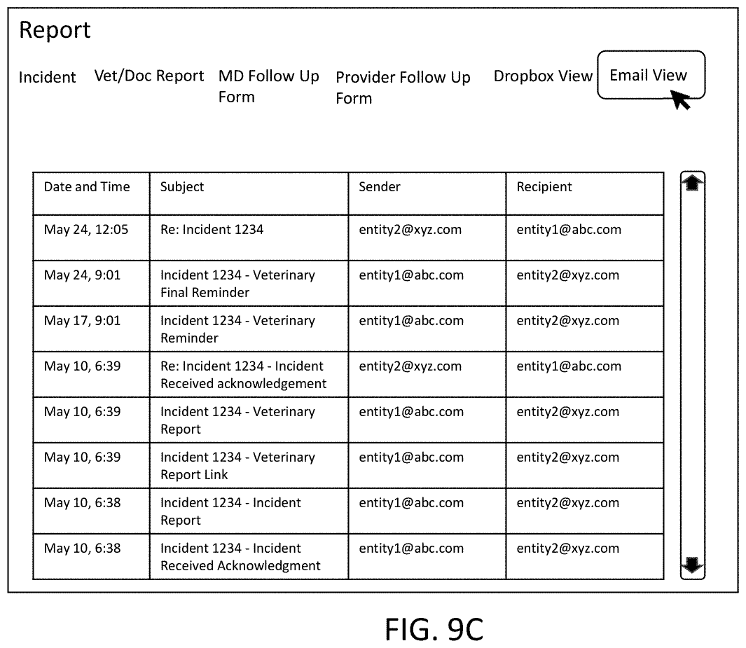

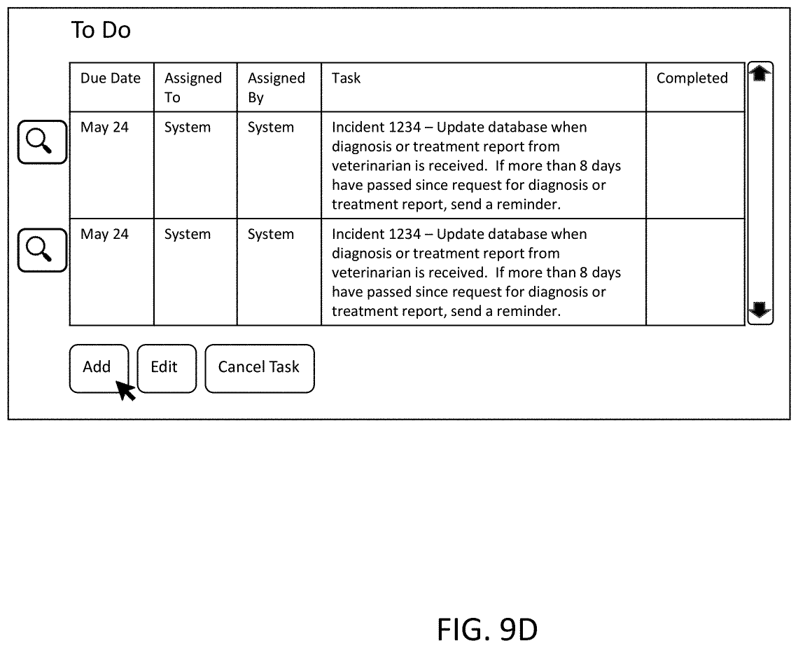

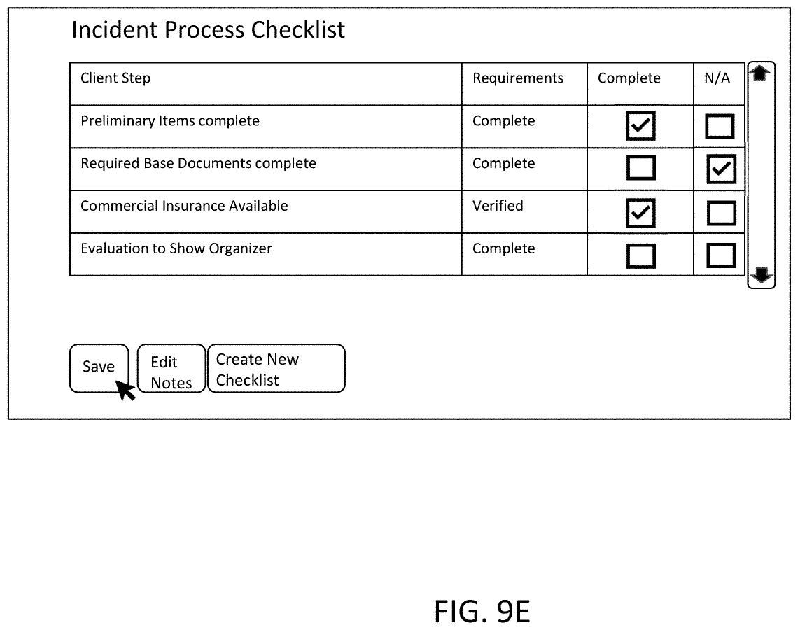

In these examples, the incident management system 110 may be configured to generate user interface content that includes the information relating to the incident reports and/or the task list, and to send the user interface content to the reviewer user device. For example, FIG. 9A illustrates the incident management system 110 generating content for the incident review user interface, and sending the content to the reviewer user device 145, which may display the incident review user interface for a reviewer. In an embodiment, the incident management system may be configured to generate a checklist to be reviewed as part of handling the incident report, and may cause the checklist to be included as part of the review user interface by sending user interface content associated with the checklist to a reviewer user device of a reviewer who is to handle the incident report. As discussed below, the incident review user interface may further be used to present tasks which the reviewer should perform as part of handling an incident report, and to receive from the reviewer a payment decision recommendation or payment amount recommendation regarding the incident report.

In an embodiment, the incident review user interface may present information regarding incident reports received by the incident management system 100, a task list associated with handling the incident reports, and/or a checklist associated with handling the incident reports. More particularly, the incident management system 110 may be configured to generate user interface content for the incident review user interface. In this example, the content may include multiple views that present various information, such as a first view that presents a list of incident reports received by the incident management system, a second view that presents data files which are associated with the incident reports and have been received by the incident management system, a third view that presents messages which are associated with the incident report and have been sent to or received by the incident management system, and/or a fourth view that presents information (e.g., treatment report or discharge report) received by the incident management system 110 from a medical service provider 120. FIGS. 9B and 9C present an example of an incident review user interface that provides the view for listing incident reports, a view for presenting diagnosis or treatment information from a medical service provider (e.g., veterinarian or doctor), a view that identifies tasks associated with following up with the medical service provider, a view associated with following up with another provider, a view for presenting files associated with the incident report (which may be uploaded to, e.g., Dropbox®), and a view associated with e-mails that have been received or sent by the incident management system 110 in connection with the incident report.

FIG. 9D illustrates an example in which the incident review user interface presents a task list. In this example, the incident review user interface may describe a list of tasks, a due date for each of the tasks, a reviewer or system (e.g., incident management system 110) assigned to the task, a reviewer or system which is assigning the task, a description of the task, and a date on which the task is completed. FIG. 9E illustrates an example in which the incident review user interface presents a checklist associated with handling an incident report. The checklist may include issues to be checked as part of handling the incident report. For instance, the issues may include whether preliminary items of information for processing the incident report are complete, whether required base documents (e.g., treatment report) for processing the incident report have been received, whether verification has been made regarding if the registrant has a commercial insurance policy, and whether an evaluation of the incident report has been made to the show organizer.

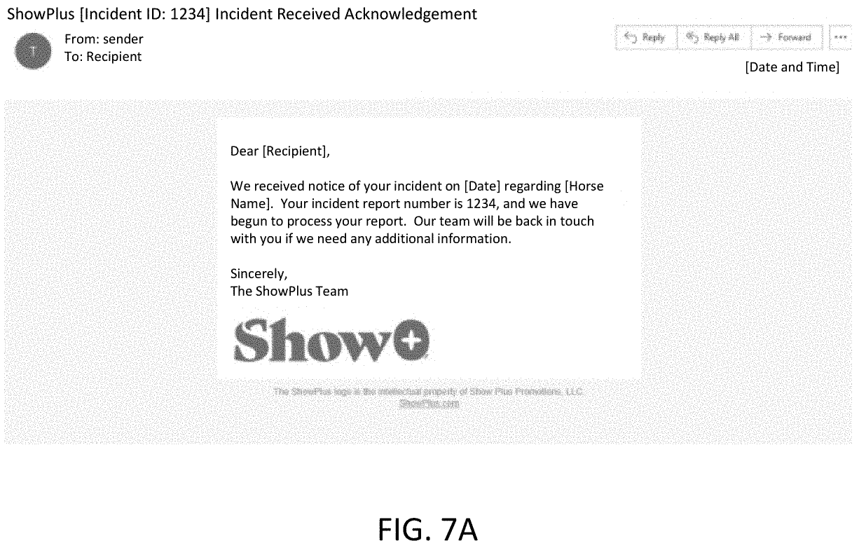

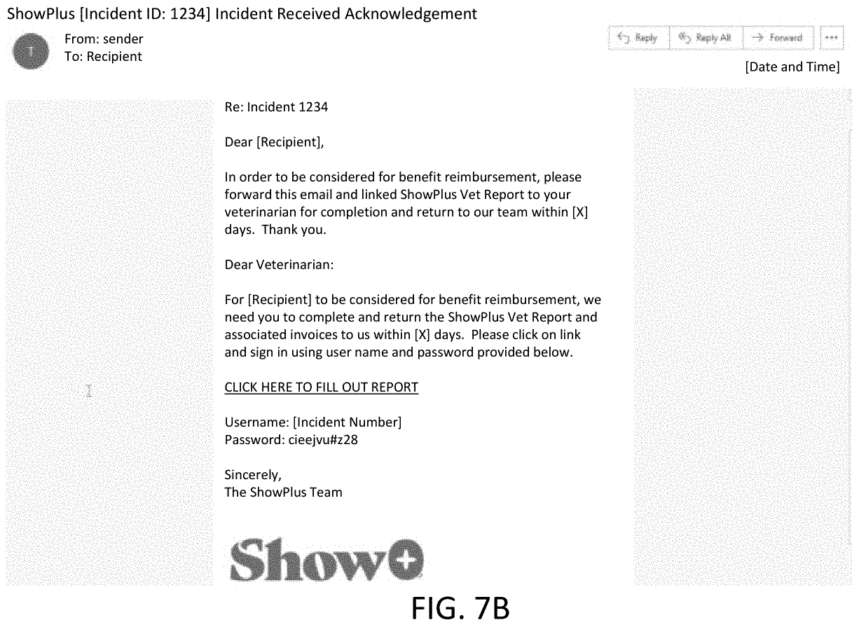

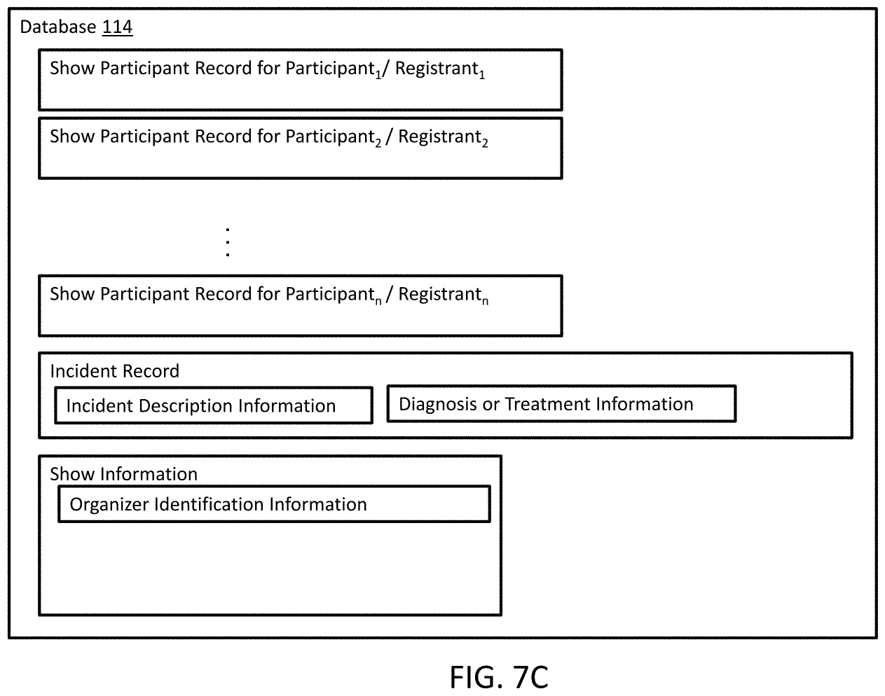

Returning to FIGS. 3A and 3B, the method 300 may include a step 304 in which the incident management system 110 provides acknowledgment to the registrant user device 150. For example, the incident management system 110 may send an acknowledgment e-mail, such as the one in FIG. 7A, to the registrant user device 150. In some instances, the acknowledgment step may include requesting diagnosis or treatment information associated with treating an incident. More particularly, the incident management system 110 may generate an information request message (e.g., e-mail) that includes a request for diagnosis or treatment information associated with the incident described in the incident report. In some instances, the information request message may be generated in response to the incident report passing the report verification operation, or more specifically in response to the horse or rider in the incident report matching one of the horses or riders in the show participant information. FIG. 7B depicts an example of an information request message, or more specifically e-mail, which the incident management system 110 may generate and send (e.g., via the communication sub-system 116) to the registrant user device 150. As depicted in FIG. 7B, the diagnosis or treatment information may be requested from a medical service provider that has provided or will provide treatment for the incident in the incident report. More particularly, the diagnosis or treatment information is requested from the medical service provider that will treat or diagnose the horse or rider involved in the incident, or that has treated or diagnosed the horse or rider involved in the incident.

In an embodiment, the information request message may include instructions for how the medical service provider can provide the diagnosis or treatment information to the incident management system. For instance, FIG. 7B depicts an example in which the information request message includes a link (e.g., a uniform resource locator, or URL) which a user (e.g., personnel at the medical service provider) may click to navigate to a website at which the user can provide the diagnosis or treatment information. In some implementations, the website may be part of the incident reporting user interface illustrated in FIG. 1B and FIGS. 5A-5C. The information request message in this example may include login information, such as a username and password, for the medical service provider. The login information may be usable by the medical service provider for access the website, wherein the diagnosis or treatment information may be received by the incident management system 110 via the website.

In an embodiment, the incident management system 110 may send the information request message to the registrant user device 150, and the device 150 may forward the information request message to the medical service provider system 120, as depicted in FIG. 3B. The information request message may be received by a user (e.g., personnel) operating or otherwise associated with the medical service provider system 120. As stated above, the information request message may include a link for accessing the incident reporting user interface, or some other user interface for providing the diagnosis or treatment information. When the personnel or other user clicks on the link, the medical service provider system 120 may communicate with the incident management system 110 to access the incident reporting user interface. For instance, the incident management system 110 may prompt the personnel or other user at the medical service provider for login information, which the personnel or other user may enter in response to the prompt. When the user enters the login information, the incident management system 110 may verify whether the login information is valid. If the login information is valid, the incident management system 110 may send web content for the incident reporting user interface to the medical service provider system 120, which may display the incident reporting user interface. The incident reporting user interface may include, e.g., a form which seeks diagnosis or treatment information, or other information which is specifically to be provided by the medical service provider. More particularly, the incident management system 110 may generate web content that includes the form, and send the content to the user's device, which may be, e.g., part of the medical service provider system 120. The web content may cause the user's device to display a webpage having the form, through which the user may enter diagnosis or treatment information after the medical service provider has diagnosed or treated the horse or rider for the incident.

More particularly, as illustrated in FIGS. 3A and 3B, the method 300 may include a step 305 in which the incident management system 110 receives, after a medical service provider has diagnosed or treated a horse or rider for the incident described in the incident report, the requested diagnosed or treatment information from a medical service provider system 120 of the medical service provider. The diagnosis or treatment information may describe, e.g., a type of injury suffered by the horse or rider, a location of the injury on a body of the horse or rider, a severity of the injury, and/or treatment (if any) that was administered by the medical service provider for the injury. In some implementations, the diagnosis or treatment information may indicate whether it is the rider or the horse who suffered an injury, whether the injury to the rider resulted in death, or whether the injury to the horse resulted in the horse being euthanized. As stated above, the incident management system 110 may receive the diagnosis or treatment information via the communication sub-system 116, or more specifically via the incident reporting user interface provided by the communication sub-system 116. In some instances, if the medical service provider system 120 includes a computing device such as an e-mail server or a database server, the diagnosis or treatment information may be received from the e-mail server or database server as an e-mail or database file. In some instances, if the medical service provider system 120 includes a facsimile (fax) machine, the diagnosis or treatment information may be received via fax.

In an embodiment, step 305 may further include storing the diagnosis or treatment information in the storage sub-system 117 of the incident management system 110, or more specifically in the database 114 provided on the storage sub-system 117. The database 114 may be managed by the database module 117a, which may store the diagnosis or treatment information in a manner that associates the information with the incident report. For instance, FIG. 7C depicts an example in which the incident report is stored as an incident record, and in which the diagnosis or treatment information for the incident described in the incident report is stored as part of the same incident record.

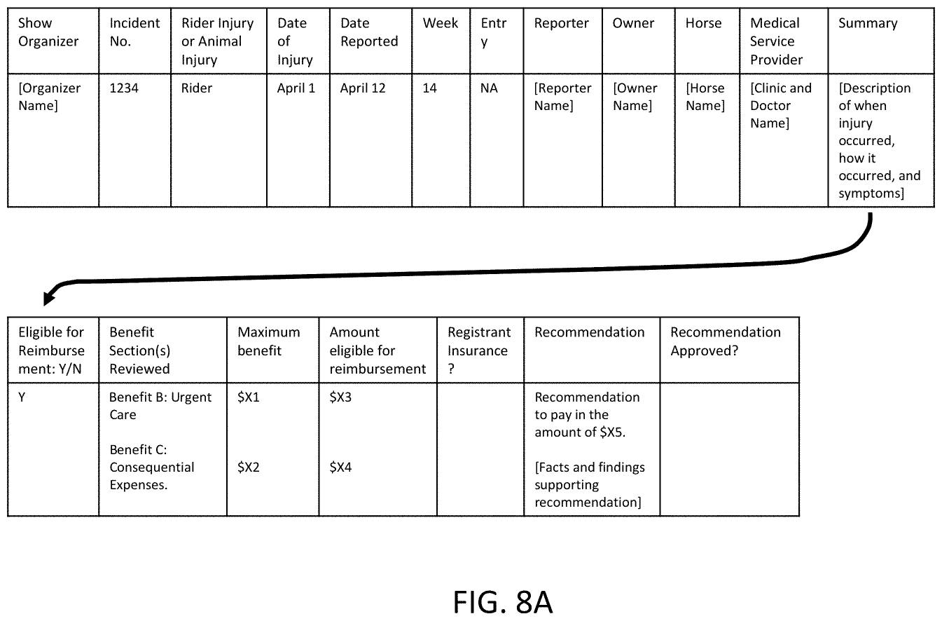

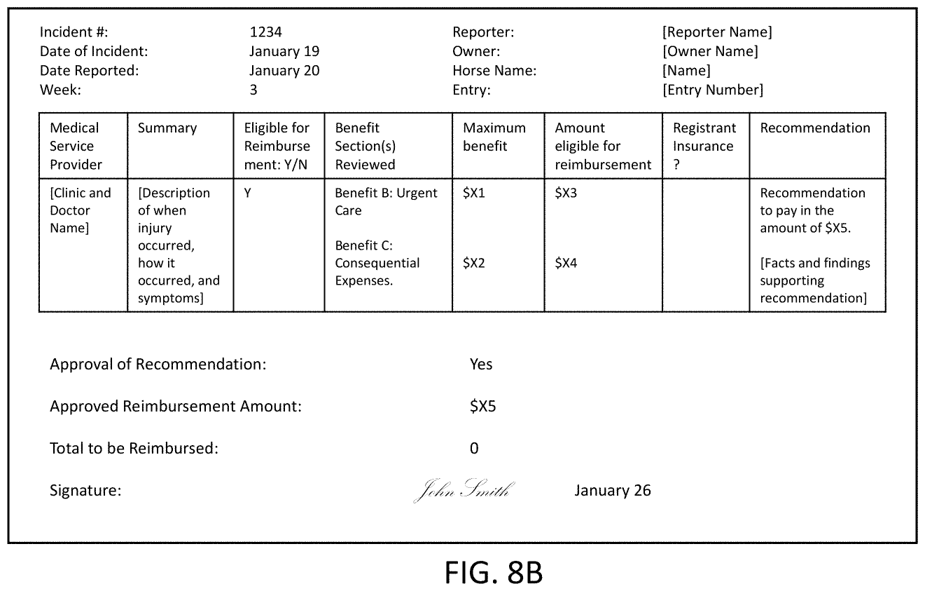

Returning to FIGS. 3A and 3B, the method 300 may in an embodiment include step 306, in which the incident management system 110 may send information associated with an incident report to the show organizer, or more specifically to the show organizer system 130 via the communication sub-system. For example, the information may be from the database 114, and more specifically may be part of an incident record that describes a specific incident. FIG. 8A illustrates an example of the information, or more specifically a spreadsheet, that the incident management system 110 may send to the show organizer. The spreadsheet may be sent, e.g., as part of an e-mail from the incident management system 110 to a computing device or other component of the show organizer system 130. In one example, this information may describe, e.g., a type of injury in an incident described by an incident report, a date of the incident, the date the incident was reported, a horse name and/or rider name of the horse or rider involved in the incident, a medical service provider which diagnosed or treated the incident, and a summary of the incident, including a summary of the injury which occurred as part of the incident. In one example, as also illustrated in FIG. 8A, this information may include payment recommendation information, which is discussed below, that recommends a possible payment of a benefit arising out of receipt of an incident report. The payment may be for a cost or loss associated with an injury or death in a reported incident. The spreadsheet may include information relating to only a single incident report, or may have a group view that includes information for multiple incident reports. As stated above, the show organizer system 130 may be a computing system that includes, e.g., an e-mail server and/or other computing devices (e.g., desktop computers, laptops, facsimile machines). For instance, if the show organizer system 130 includes an e-mail server and/or a laptop which is able to view e-mails, then the incident management system 110 may be configured to send the information relating to the incident as an e-mail to the e-mail server and/or laptop of the show organizer system 130. Personnel of the show organizer may be able to view the e-mail via the laptop or other component of the show organizer system 130.

In an embodiment, the method 300 may include a step 307, in which the incident management system 110 may receive, from the show organizer system 130, a benefit payment decision indication (also referred to as a payment decision indication) which indicates approval or denial of payment with respect to an incident described in the incident report. For example, FIG. 8B illustrates a document or other information received by the incident management system 110 from the show organizer, or more specifically the show organizer system 130. The document may include an indication that payment for a particular reported incident is approved. In this embodiment, the payment may be made under a benefits plan provided by the show organizer, which may be eligible for reimbursement with a captive associated with the show organizer. For instance, after the show organizer system 130 receives information that describes an incident in an incident report, an equine show organizer may use the information to make a determination of whether to pay a benefit associated with the incident in accordance with a benevolent benefits plan. After the show organizer makes such a decision, it may provide a notification of the decision to the incident management system 110. Thus, the incident management system 110 may, in step 307 receive the payment decision indication from the show organizer 130 via the communication sub-system 116, which may receive the payment decision indication via, e.g., a network such as the Internet.

In an embodiment, the payment decision indication may be based on payment recommendation information from the incident management system 110. More specifically, the incident management system 110, or more specifically the processing sub-system 111 thereof, may be configured to send payment recommendation information to the show organizer, or more specifically to the show organizer system 130, as illustrated in FIG. 9A. The recommendation to approve or deny a benefit payment may indicate whether the incident described by the incident report is eligible for receipt of a benefit payment and in a determined amount. The organizer of the equine show may generate the benefit payment decision indication based on the payment recommendation information, and send the benefit payment decision indication to the incident management system 110.

In some instances, the benefit payment recommendation information for an incident report may be based on information generated or determined by a reviewer of the incident report. More particularly, the reviewer may review the incident report, and formulate a recommendation, and send benefit payment recommendation to the incident management system 110 (e.g., via the incident review user interface). In an embodiment, the benefit payment recommendation information may be based on a determination (e.g., by the reviewer of the incident report) of what is a reasonable expense for treating an injury described in the incident report, wherein a recommended amount of benefits may be equal or proportional to the determined reasonable expense. The incident management system may then send the benefit payment recommendation information to the show organizer.

Returning to FIGS. 3A and 3B, the method 300 may in an embodiment include a step 308, in which the incident management system 110 generates and/or sends a status notification message that indicates whether the registrant which reported a particular incident will receive payment associated with the incident. More particularly, the incident management system 110 may send the status notification message to a registrant user device 150 of a registrant that submitted the incident report, wherein the notification message may be sent via the communication sub-system 116 over a network (e.g., the Internet). The status notification message may be generated based on the benefit payment decision indication and/or benefit payment recommendation information. For example, if the benefit payment decision indication from the show organizer 130 indicates denial of payment for the incident, the status notification message generated by the incident management system may also indicate that payment for the incident under the benefits plan has been denied. If the benefit payment decision indication from the show organizer 130 indicates approval of payment of a benefit for the incident, the status notification message from the incident management system 110 may also indicate that payment under the benefits plan has been approved for the incident. As stated above, the payment may be made by the equine show organizer, which may be eligible for reimbursement by a captive associated with the equine show organizer. In some instances, if the incident management system 110 is operated by an entity that is acting as an administrator of the benefits plan discussed above, this entity may advance the payment to the registrant for a particular reported incident, and then seek reimbursement for the advanced payment from the show organizer. In such instances, the show organizer may reimburse the administrator entity, and then may itself seek reimbursement from a captive which has a policy covering the show organizer. While FIG. 3 illustrates various steps of one embodiment of method 300, other embodiments of the method may omit one or more of the steps in FIG. 3. For instance, the method 300 may omit steps 301 and 303.

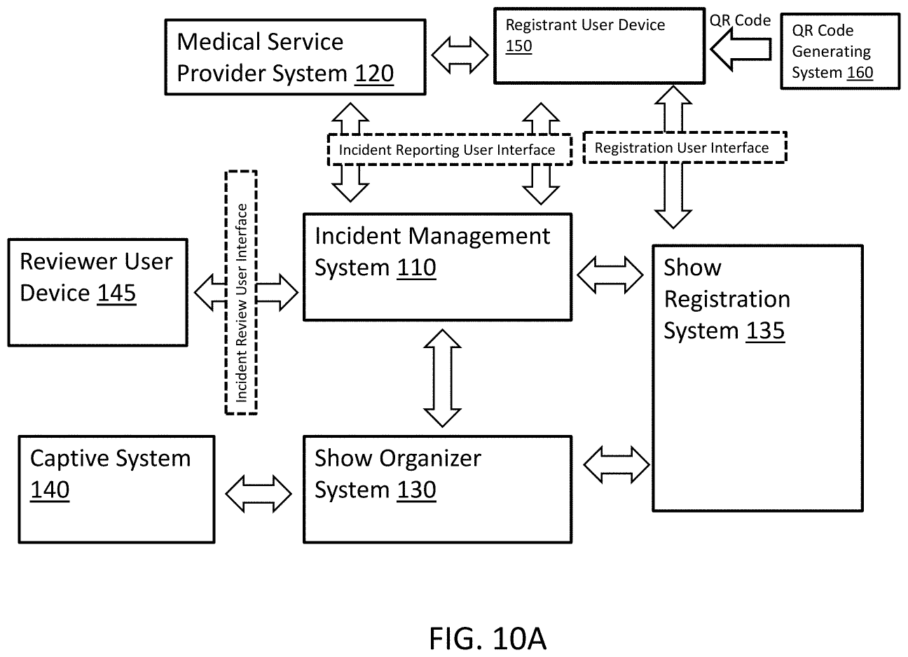

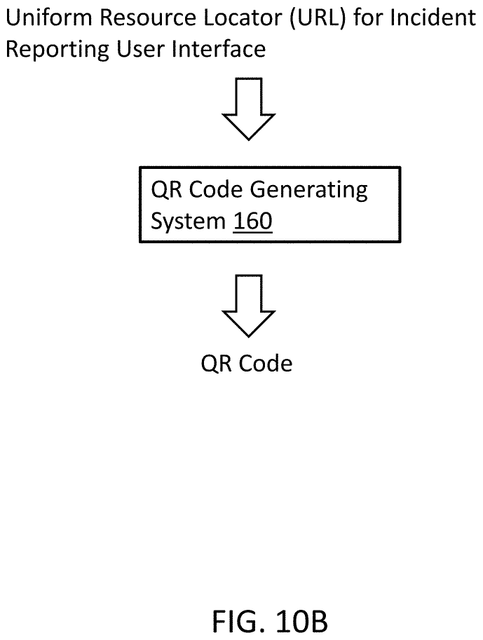

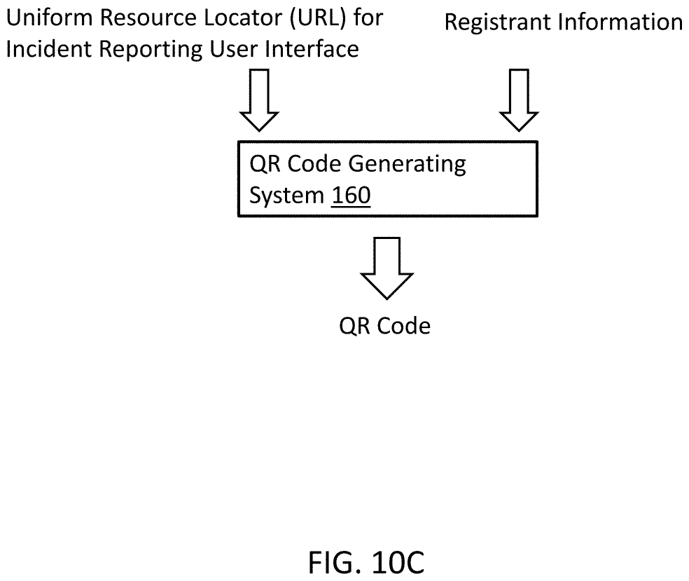

FIG. 10A illustrates an example that includes the components of FIG. 1D, and further includes a QR code generating system 160. The QR code generating system 160 may facilitate user access to an incident reporting user interface. For instance, the QR code generating system 160 may be configured to generate a QR code that encodes an online address (e.g., a uniform resource locator, or URL) of the incident reporting user interface. In some cases, the generated QR code may encode information specific to the registrant, such as horse identification information or rider identification information, which may simplify incident reporting for the registrant. More specifically, FIGS. 10B and 10C depict operation of the QR code generating system 160, which may be configured to generate a QR code that simplifies access of a web page or web portal for submitting an incident report. To simplify access to the web page, the QR code generating system 160 may be configured to generate a QR code which encodes a uniform resource locator (URL) or other identifier of the web page. A registrant may be able to access the web page by simply scanning the QR code. If the registrant is a person, the scanning may be performed by that person using his or her registrant user device. If the registrant is an organization, the scanning may be performed by personnel of the organization using a registrant user device. In both cases, the scanning may allow the registrant to avoid manually inputting the URL. In some implementations, as depicted in FIG. 10C, the QR code generating system 160 can encode certain registrant-specific information into the QR code. For example, the system 160 may be configured to encode a back number or chip number of a horse into the QR code, such as via a URL variable. Such a QR code may further simplify submission of an incident report, because the incident management system 110 may be configured to automatically extract certain information (e.g., horse identification information) from such a QR code.

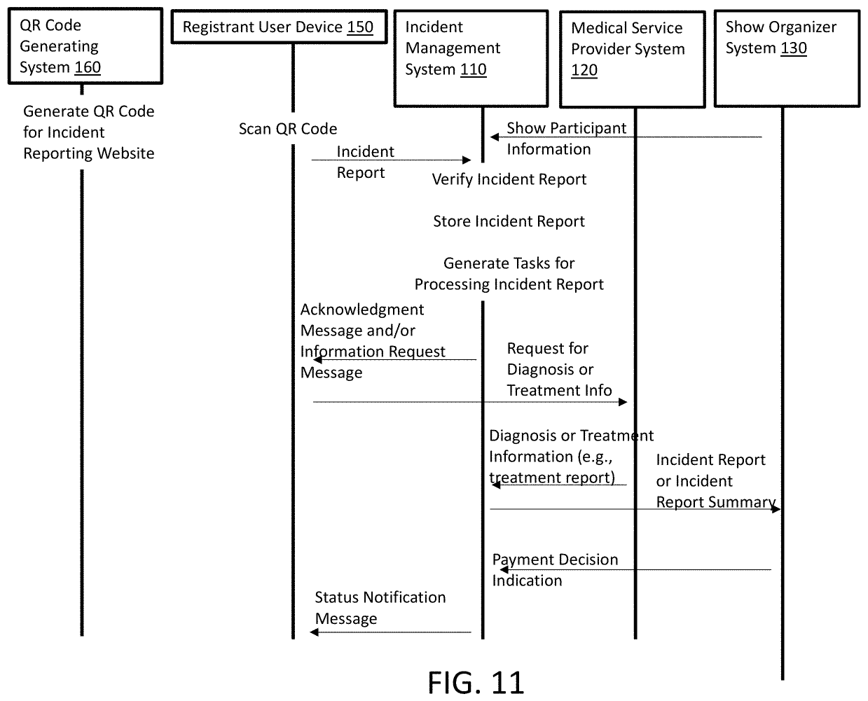

FIG. 11 provides a diagram which depicts an example interaction among components of system in FIG. 10A. In this example, the QR code generating system 160 may generate a QR code which encodes a URL for accessing an incident reporting website. The registrant user device 150 may scan the QR code to access the incident reporting website, and to submit an incident report to the incident management system 110.

### Additional Discussion of Various Embodiments

Embodiment 1 of the present disclosure relates to an incident management system for managing incident reporting for an equine show. The incident management system may comprise a communication sub-system, a storage sub-system, and a processing sub-system. The communication sub-system may be configured to receive information from or send information to: (i) a show organizer system or show registration system associated with an organizer of the equine show, (ii) registrant user devices of registrants are associated with horses that are participating or have participated in the equine show, and (iii) medical service provider systems of medical service providers. The processing sub-system may be in communication with the storage sub-system and the communication sub-system, and may have one or more processing circuits configured to: receive via the communication sub-system, from the show organizer system or the show registration system, show participant information which identifies horses or riders registered to participate in the equine show; store the show participant information in a database provided on the storage sub-system; receive via the communication sub-system, from a registrant user device of a registrant, an incident report for an incident in which a horse or a rider associated with the registrant suffered an injury or death, wherein the incident report includes incident description information which describes the incident, and wherein the registrant user device is one of the registrant user devices; store the incident report in the database provided on the storage sub-system; perform a report verification operation by comparing the horse or rider described in the incident report with the show participant information to determine whether the horse or the rider in the incident report matches one of the horses or riders identified in the show participant information; generate, in response to a determination that the horse or rider in the incident report matches one of the horses or riders in the show participant information, an information request message which includes a request for diagnosis or treatment information associated with the incident, wherein the diagnosis or treatment information is requested from a medical service provider that will treat or diagnose the horse or rider for the incident, or that has treated or diagnosed the horse or rider for the incident; send the information request message to the registrant user device via the communication sub-system; receive via the communication sub-system, after the medical service provider has diagnosed or treated the horse or rider for the incident, the diagnosis or treatment information from a medical service provider system of the medical service provider; store the diagnosis or treatment information in the database in a manner that associates the diagnosis or treatment information with the incident report; send, to the show organizer via the communication sub-system, information from the database that is associated with the incident report; payment decision indication which indicates approval or denial of payment for a cost or loss associated with the incident; and generate, based on the payment decision indication, a status notification message which indicates whether the registrant will receive payment for the cost or loss associated with the incident.

Embodiment 2 includes the incident management system of embodiment 1, wherein the communication sub-system is further configured to communicate with one or more reviewer user devices of one or more reviewers of incident reports. The processing sub-system in this embodiment is configured to: generate, in response to receiving the incident report, a task list which includes a plurality of tasks associated with handling the incident report; generate user interface content for an incident review user interface, wherein the incident review user interface is a graphical user interface (GUI) for presenting incident reports that have been received by the incident management system, and is further for presenting the task list; and send, via the communication sub-system, the user interface content to the one or more reviewer user devices, to cause the one or more reviewer user devices to display the incident review user interface.

Embodiment 3 includes the incident management system of embodiment 2, wherein the processing sub-system is configured to: assign each task on the task list to: (i) the one or more reviewers or (ii) the incident management system, determine, periodically, whether the task list includes an unfulfilled task and whether a condition for performing the unfulfilled task is satisfied. The processing sub-system is configured to, in response to a determination that the task list includes the unfulfilled task and that the condition for performing the unfulfilled task is satisfied, determine whether the unfulfilled task is assigned to the incident management system or to the one or more reviewers; is configured to, in response to a determination that the unfulfilled task is assigned to the incident management system, perform the unfulfilled task; and is configured to, in response to a determination that the unfulfilled task is assigned to the one or more reviewers, send a task reminder message to one or more reviewer user devices of the one or more reviewers.

Embodiment 4 includes the incident management system of embodiment 2 or 3, wherein the plurality of tasks in the task list includes an information request reminder task assigned to the incident management system, wherein a condition for performing the information request reminder task includes the incident management system not having received the diagnosis or treatment information from the medical service provider, and includes a deadline for receiving the diagnosis or treatment information being within a predefined amount of time from passing, wherein the processing sub-system is configured, in response to a determination that the condition for performing the information request reminder task has been satisfied, to perform the information request reminder task by sending an information request reminder message to the registrant user device.

Embodiment 5 includes the incident management system of any one of embodiments 2-4, wherein the user interface content generated by the processing sub-system includes: content for a first view that presents a list of incident reports received by the incident management system, content for a second view that presents data files which are associated with the incident reports and have been received by the incident management system, and content for a third view that presents messages which are associated with the incident report and have been sent or received by the incident management system.

Embodiment 6 includes the incident management system of embodiment 5, wherein the user interface content generated by the processing sub-system further includes content for one or more additional views that present information received by the incident management system from the medical service provider system.

Embodiment 7 includes the incident management system of any one of embodiments 2-6, wherein the processing sub-system is configured to generate, in response to receiving the incident report, a checklist to be reviewed as part of handling the incident report, wherein the user interface content sent to the one or more reviewer user devices causes the one or more reviewer user devices to display the checklist on the incident review user interface.

Embodiment 8 includes the incident management system of any one of embodiments 1-7, wherein the processing sub-system is configured to generate user interface content for an incident reporting user interface, wherein the incident reporting user interface is a GUI having a plurality of form fields for data entry, wherein the incident report is received from the registrant via the incident reporting user interface.

Embodiment 9 includes the incident management system of embodiment 8, wherein the processing sub-system is configured to include, in the information request message, login information which is usable by the medical service provider for accessing the incident reporting user interface, wherein the diagnosis or treatment information is received from the medical service provider via the incident reporting user interface.

Embodiment 10 includes the incident management system of embodiment 8 or 9, wherein the processing sub-system is configured, during the report verification operation for the incident report, to determine whether the incident report received via the incident reporting user interface is missing information in at least one of the form fields; and to send, in response a determination that the incident report is missing information in at least one of the form fields, a request to the registrant user device to provide the information that is missing.

Embodiment 11 includes the incident management system of any one of embodiments 1-10. The processing sub-system is further configured to: send, to the show organizer system via the communication sub-system, payment recommendation information which indicates whether the incident described by the incident report should be approved or denied payment for the cost or loss associated with the incident, and/or indicates an amount of the payment, wherein the incident management system receives the payment decision indication from the show organizer system after the incident management system has sent the payment recommendation information to the show organizer system.

Embodiment 12 includes the incident management system of embodiment 11, wherein the payment recommendation information includes a benefit recommendation and/or a benefit payment amount recommendation that is received by the incident management system from a reviewer user device of a reviewer of the incident report.

Embodiment 13 includes the incident management system of any one of embodiments 1-12, wherein the processing sub-system is further configured to receive and store, in the database, benefit plan information which describes a benefit plan provided by the organizer for the equine show for payments associated with incidents at the equine show.

Embodiment 14 includes the incident management system of embodiment 13, wherein the processing sub-system is configured to generate reimbursement request information which the show organizer is able to use, after providing payment under the benefit plan to a registrant, to seek reimbursement for the payment via submission of a claim with a captive associated with the organizer of the equine show.

Embodiment 14 includes the incident management system of embodiment 1, wherein the incident report indicates includes horse identification information, rider identification information, and provider identification information, wherein the horse identification information identifies the horse associated with the registrant, wherein the rider identification information identifies the rider associated with the registrant, and wherein the partner identification information identifies a medical service provider that will treat or has treated an injury in the incident report.

Embodiment 15 includes the incident management system of embodiment 14, wherein the horse identification information comprises at least one of: a chip number associated with the horse or a back number assigned to the horse, and wherein the rider information comprises a name of the rider.

Embodiment 18 includes the incident management system of any one of embodiments 1-17, wherein the benefit payment decision is for diagnosing or treating a rider injury, for satisfying deductibles and out-of-pocket maximums as part of diagnosing or treating the rider injury, for ongoing household or business-related expenses arising from the rider injury, for diagnosing or treating a horse injury, for transportation costs associated with treating the rider injury or the horse injury, for death of the horse due to the horse injury.

Embodiment 19 includes the incident management system of any one of embodiments 1-18, wherein if the injury in the incident report causes the rider and/or the horse to be unable to continue participating in the equine show, the benefit plan may provide for a refund of an entrance fee paid by the registrant for registering to participate in the equine show.

While various embodiments have been described above, it should be understood that they have been presented by way of example only, and not limitation. Where methods described above indicate certain events occurring in certain order, the ordering of certain events may be modified. Additionally, certain of the events may be performed concurrently in a parallel process when possible, as well as performed sequentially as described above.

Where schematics and/or embodiments described above indicate certain components arranged in certain orientations or positions, the arrangement of components may be modified. While the embodiments have been particularly shown and described, it will be understood that various changes in form and details may be made. Any portion of the apparatus and/or methods described herein may be combined in any combination, except mutually exclusive combinations. The embodiments described herein can include various combinations and/or sub-combinations of the functions, components, and/or statistical models of the different embodiments described.

What is claimed is:

1. An incident management system for managing incident reporting for an equine show, the incident management system comprising:
a communication sub-system configured to receive information from or send information to: (i) a show organizer system or show registration system associated with an organizer of the equine show, (ii) registrant user devices of registrants associated with horses that are participating or have participated in the equine show, and (iii) medical service provider systems of medical service providers;
a storage sub-system; and
a processing sub-system in communication with the storage sub-system and the communication sub-system, and having one or more processing circuits configured to:
receive via the communication sub-system, from the show organizer system or the show registration system, show participant information which identifies horses or riders registered to participate in the equine show;
store the show participant information in a database provided on the storage sub-system;
receive via the communication sub-system, from a registrant user device of a registrant of the equine show, an incident report for an incident in which a horse or a rider associated with the registrant suffered an injury or death, wherein the incident report includes incident description information which describes the incident, and wherein the registrant user device is one of the registrant user devices with which the communication sub-system is configured to communicate;
store the incident report in the database provided on the storage sub-system;
perform a report verification operation by comparing the horse or rider described in the incident report with the show participant information to determine whether the horse or the rider in the incident report matches one of the horses or riders identified in the show participant information;
generate, in response to a determination that the horse or rider in the incident report matches one of the horses or riders in the show participant information, an information request message which includes a request for diagnosis or treatment information associated with the incident, wherein the diagnosis or treatment information is requested from a medical service provider that will treat or diagnose the horse or rider for the incident, or that has treated or diagnosed the horse or rider for the incident;
send the information request message to the registrant user device via the communication sub-system;
receive via the communication sub-system, after the medical service provider has diagnosed or treated the horse or rider for the incident, the diagnosis or treatment information from a medical service provider system of the medical service provider;
store the diagnosis or treatment information in the database in a manner that associates the diagnosis or treatment information with the incident report;
send, to the show organizer system via the communication sub-system, information from the database that is associated with the incident report;
receive via the communication system, from the show organizer system, a payment decision indication which indicates approval or denial of payment for a cost or loss associated with the incident; and
generate, based on the payment decision indication, a status notification message which indicates whether the registrant will receive payment for the cost or loss associated with the incident; and
send the status notification message to the registrant user device via the communication sub-system.

2. The incident management system of claim 1, wherein the communication sub-system is further configured to communicate with one or more reviewer user devices of one or more reviewers of incident reports,
wherein the processing sub-system is configured to:
generate, in response to receiving the incident report, a task list which includes a plurality of tasks associated with handling the incident report;
generate user interface content for an incident review user interface, wherein the incident review user interface is a graphical user interface (GUI) for presenting incident reports that have been received by the incident management system, and is further for presenting the task list; and
send, via the communication sub-system, the user interface content to the one or more reviewer user devices, to cause the one or more reviewer user devices to display the incident review user interface.

3. The incident management system of claim 2, wherein the processing sub-system is configured to:
assign each task on the task list to: (i) the one or more reviewers or (ii) the incident management system,
determine, periodically, whether the task list includes an unfulfilled task and whether a condition for performing the unfulfilled task is satisfied,
in response to a determination that the task list includes the unfulfilled task and that the condition for performing the unfulfilled task is satisfied, determine whether the unfulfilled task is assigned to the incident management system or to the one or more reviewers,
in response to a determination that the unfulfilled task is assigned to the incident management system, perform the unfulfilled task,
in response to a determination that the unfulfilled task is assigned to the one or more reviewers, send a task reminder message to one or more reviewer user devices of the one or more reviewers.

4. The incident management system of claim 3, wherein the plurality of tasks in the task list includes an information request reminder task assigned to the incident management system,
wherein a condition for performing the information request reminder task includes the incident management system not having received the diagnosis or treatment information from the medical service provider, and includes a deadline for receiving the diagnosis or treatment information being within a predefined amount of time from passing,
wherein the processing sub-system is configured, in response to a determination that the condition for performing the information request reminder task has been satisfied, to perform the information request reminder task by sending an information request reminder message to the registrant user device.

5. The incident management system of claim 2, wherein the user interface content generated by the processing sub-system includes: content for a first view that presents a list of incident reports received by the incident management system, content for a second view that presents data files which are associated with the incident reports and have been received by the incident management system, and content for a third view that presents messages which are associated with the incident report and have been sent or received by the incident management system.

6. The incident management system of claim 5, wherein the user interface content generated by the processing sub-system further includes content for one or more additional views that present information received by the incident management system from the medical service provider system.

7. The incident management system of claim 2, wherein the processing sub-system is configured to generate, in response to receiving the incident report, a checklist to be reviewed as part of handling the incident report, wherein the user interface content sent to the one or more reviewer user devices cause the one or more reviewer user devices to display the checklist on the incident review user interface.

8. The incident management system of claim 1, wherein the processing sub-system is configured to generate user interface content for an incident reporting user interface, wherein the incident reporting user interface is a GUI having a plurality of form fields for data entry, wherein the incident report is received from the registrant via the incident reporting user interface.

9. The incident management system of claim 8, wherein the processing sub-system is configured to include, in the information request message, login information which is usable by the medical service provider for accessing the incident reporting user interface,
wherein the diagnosis or treatment information is received from the medical service provider via the incident reporting user interface.

10. The incident management system of claim 8, wherein the processing sub-system is configured, during the report verification operation for the incident report, to determine whether the incident report received via the incident reporting user interface is missing information in at least one of the form fields, and to send, in response a determination that the incident report is missing information in at least one of the form fields, a request to the registrant user device to provide the information that is missing.

11. The incident management system of claim 1, wherein the processing sub-system is further configured to:
send, to the show organizer system via the communication sub-system, payment recommendation information which indicates whether the incident described by the incident report should be approved or denied payment for the cost or loss associated with the incident, and/or indicates an amount of the payment,
wherein the incident management system receives the payment decision indication from the show organizer system after the incident management system has sent the payment recommendation information to the show organizer system.

12. The incident management system of claim 11, wherein the payment recommendation information includes a benefit recommendation and/or a benefit payment amount recommendation that is received by the incident management system from a reviewer user device of a reviewer of the incident report.

13. The incident management system of claim 1, wherein the processing sub-system is further configured to receive and store, in the database, benefit plan information which describes a benefit plan provided by the organizer for the equine show for payments associated with incidents at the equine show.

14. The incident management system of claim 13, wherein the processing sub-system is configured to generate reimbursement request information which the show organizer is able to use, after providing payment under the benefit plan to a registrant, to seek reimbursement for the payment via submission of a claim with a captive associated with the organizer of the equine show.

15. An incident management system for managing incident reporting for an equine show, the incident management system comprising:
a communication sub-system configured to receive information from or send information to: (i) a show organizer system or show registration system associated with an organizer of the equine show, (ii) registrant user devices of registrants associated with horses that are participating or have participated in the equine show, and (iii) medical service provider systems of medical service providers;
a storage sub-system; and
a processing sub-system in communication with the storage sub-system and the communication sub-system, and having one or more processing circuits configured to:
receive via the communication sub-system, from a registrant user device of a registrant, an incident report for an incident in which a horse or a rider associated with the registrant suffered an injury or death, wherein the incident report includes incident description information which describes the incident, and wherein the registrant user device is one of the registrant's user devices;
store the incident report in a database provided on the storage sub-system;
generate an information request message which includes a request for diagnosis or treatment information associated with the incident, wherein the diagnosis or treatment information is requested from a medical service provider that will treat or diagnose the horse or rider for the incident, or that has treated or diagnosed the horse or rider for the incident;
send the information request message to the registrant user device via the communication sub-system;
receive via the communication sub-system, after the medical service provider has diagnosed or treated the horse or rider for the incident, the diagnosis or treatment information from a medical service provider system of the medical service provider;
store the diagnosis or treatment information in the database in a manner that associates the diagnosis or treat information with the incident report;
send, to the show organizer system via the communication sub-system, information from the database that is associated with the incident report;
receive via the communication system, from a show organizer system associated with an organizer of the equine show, a payment decision indication which indicates approval or denial of payment for a cost or loss associated with the incident; and
generate, based on that payment decision indication, a status notification message which indicates whether the registrant will receive payment for the cost or loss associated with the incident; and
send the status notification message to the registrant user device via the communication sub-system.

16. The incident management system of claim 15, wherein the communication sub-system is configured to communicate with one or more reviewer user devices of one or more reviewers of incident reports,
wherein the processing sub-system is configured to:
generate, in response to receiving the incident report, a task list which includes a plurality of tasks associated with handling the incident report;
generate user interface content for an incident review user interface, wherein the incident review user interface is a graphical user interface (GUI) for presenting incident reports that have been received by the incident management system, and is further for presenting the task list; and
send, via the communication sub-system, the user interface content to the one or more reviewer user devices, to cause the one or more reviewer user devices to display the incident review user interface.

17. A method performed by an incident management system for managing incident reporting for an equine show, the method comprising:
receiving, from a registrant user device of a registrant, an incident report for an incident in which a horse or a rider associated with the registrant suffered an injury or death, wherein the incident report includes incident description information which describes the incident;
storing the incident report in a database provided by incident management system;
generating an information request message which includes a request for diagnosis or treatment information associated with the incident, wherein the diagnosis or treatment information is requested from a medical service provider that will treat or diagnose the horse or rider for the incident, or that has treated or diagnosed the horse or rider for the incident;
sending the information request message to the registrant user device;
receiving, after the medical service provider has diagnosed or treated the horse or rider for the incident, the diagnosis or treatment information from a medical service provider system of the medical service provider;
storing the diagnosis or treatment information in the database in a manner that associates the diagnosis or treat information with the incident report;
sending, to an organizer of the equine show, information from the database that is associated with the incident report;
receiving, from the organizer, a payment decision indication which indicates approval or denial of payment for a cost or loss associated with the incident; and
generating, based on that payment decision indication, a status notification message which indicates whether the registrant will receive payment for the cost or loss associated with the payment; and
sending the status notification message to the registrant user device.

18. The method of claim 17, further comprising:
generating, in response to receiving the incident report, a task list which includes a plurality of tasks associated with processing the incident report;
generating user interface content for an incident review user interface, wherein the incident review user interface is a graphical user interface (GUI) for presenting incident reports that have been received by the incident management system, and is further for presenting the task list; and
sending the user interface content to the one or more reviewer user devices, to cause the one or more reviewer user devices to display the incident review user interface.

19. The method of claim 18, further comprising:
assigning each task on the task list to: (i) the one or more reviewers or (ii) the incident management system,
determining, periodically, whether the task list includes an unfulfilled task and whether a condition for performing the unfulfilled task is satisfied,
in response to a determination that the task list includes the unfulfilled task and that the condition for performing the unfulfilled task is satisfied, determining whether the unfulfilled task is assigned to the incident management system or to the one or more reviewers,
in response to a determination that the unfulfilled task is assigned to the incident management system, performing the unfulfilled task by the incident management system.

20. The method of claim 18, wherein the user interface content generated by the incident management system includes: content for a first view that presents a list of incident reports received by the incident management system, content for a second view that presents data files which are associated with the incident reports and have been received by the incident management system, and content for a third view that presents messages which are associated with the incident report and have been sent or received by the incident management system.
